(19) United States
(12) Patent Application Publication
(10) Pub. No.: US 2026/0031228 A1
(43) Pub. Date: Jan. 29, 2026

(54) ORTHOBIOLOGIC IMPLEMENTATION SYSTEM

(71) Applicant: Xenco Medical, LLC, San Diego, CA (US)

(72) Inventors: Jason Haider, San Diego, CA (US); Fred Murillo, San Diego, CA (US)

(73) Assignee: Xenco Medical, LLC, San Diego, CA (US)

(21) Appl. No.: 19/286,724

(22) Filed: Jul. 31, 2025

Related U.S. Application Data
(63) Continuation-in-part of application No. 19/035,180, filed on Jan. 23, 2025, now Pat. No. 12,400,759.
Application No. 19/035,180 is a continuation of application No. 18/810,822, filed on Aug. 21, 2024.
(60) Provisional application No. 63/674,438, filed on Jul. 23, 2024.

(51) Int. Cl. G16H 40/67; A61B 5/00; A61B 5/11; A61L 27/12; A63B 71/06

(52) U.S. Cl. CPC G16H 40/67; A61B 5/0077; A61B 5/1116; A61B 5/1127; A61B 5/7465; A61L 27/12; A63B 71/0622

(57) ABSTRACT

Orthobiologics systems and methods for evaluation of exercise performance, such as physical rehabilitation performance, alone or in combination with pain remediation strategies are disclosed herein. The disclosure provides surgical inserts conducive to healing, approaches for monitoring and evaluation of surgical and nonsurgical rehabilitation, and pain management, collectively for the encouragement of rehabilitation from physical pain such as movement related physical pain.

### CROSS-REFERENCE TO RELATED APPLICATIONS

The present application is a Continuation-In-Part of U.S. Patent Application No. 19/035, 180, filed Jan. 23, 2025, which is a Continuation of U.S. patent application Ser. No. 18/810,822, filed Aug. 21, 2024, and claims the benefit of U.S. Provisional Patent Application No. 63/674,438, filed Jul. 23, 2024, the contents of each of which are hereby incorporated by reference in their entirety.

### BACKGROUND

Orthobiologics have sweeping surgical applications in treating general orthopedic and spinal pathologies. Though orthobiologics have been widely accepted as treatment for a wide number of general orthopedic and spinal pathologies, the scope of their intervention has been limited to the surgical or clinical setting. A significant gap has remained in the monitoring and amplification of these orthobiologics during the post-operative recovery phase of patients.

Ortho biotics have been produced, for example the Signafuse by Bioventus Surgical and the Vitoss spine product by Stryker. However, these products are not integrated into a rehabilitation or therapy regime so as to facilitate at home rehabilitation compliance through ready feedback, recommendations and pain management.

Similarly, pain management and exercise body monitoring have been developed, for example by Hinge Health, Sword Health, Omada Health, Kaia Health, Bardavon, Dario Health and DorsaVI.

However, to date, there have been no digitally-enabled orthobiologic technologies that enable treating physicians to link the implantation or injection of an orthobiologic, or other medical device or composition to mobility changes and physical therapy adherence, to direct patient rehab course selection or to monitor rehab adherence or efficacy, or to supplement or address challenges associated with pain management, particularly the risk of addiction associated with pharmaceutical pain management.

There is an unmet but critical need for continuity between the intraoperative or clinical treatment phases of a patient's journey and the recovery phases which is underscored by the scientifically-validated link between surgical success and physical therapy.

Additionally, though orthobiologics have been proven to be effective in pain management as both injectables and implants, there has been an unmet need to directly compound their efficacy with software-driven performance analysis technologies and pain management technologies anchored in art therapy or other mental stimulation or distraction.

### SUMMARY

Disclosed herein are orthobiologic kits, such as kits comprising one or more of an implantable orthobiologic or other medical composition or material to be applied to a subject, a pose estimation software package, a pain alleviation suite, and a physician portal software package. In some such kits, the implantable orthobiologic or other medical composition or material to be applied to a subject comprises a tag or identification code. Some implantable orthobiologics comprise one or more of bioactive glass, beta-tricalcium phosphate such as in granule form, hydroxyapatite in some cases in common granules with the beta-tricalcium phosphate, and collagen, such as bovine collagen. The implantable orthobiologic in some cases mimics kit recipient or user bone, and may be consistent with healing achieved through use of a patient autologous bone graft.

Kits often comprise at least one sticker corresponding to the tag or identification code, such that a user may be labeled according to the biologic used. Some kits comprise hermetically sealed packaging having an identification code corresponding to the tag. The tag is in some cases scannable.

The pose estimation package is often configured to interface or interfaces with one or more of a mobile image capture device, positional markers on a user, or a wearable motion sensor. The positional markers or motion sensors are variously attached to the user, such as via an adhesive or clip, or attached to the user via a stretchable band or bands, or a non-stretchable wrapping, as may be tied or attached at a suitable tightness. The pose estimation package in some cases comprises a wearable motion sensor. Alternately, the pose estimation package in some cases identifies user posture and motion without relying upon exogenous markers, in these cases identifying for example user landmarks such as joints and using these to analyze user movement data. The pose estimation package often comprises software or is otherwise operable on a computer or device having computing capacity.

The user is in some cases a surgical recipient of the implantable orthobiologic or other medical composition or material. The pose estimation package in some cases comprises a kinetic model for at least one rehabilitation exercise. The pose estimation package in some cases identifies at least one anatomical landmark in a user motion dataset. The user motion dataset may comprise at least one video frame or at least one position monitor data array, and in some cases the pose estimation package identifies the at least one anatomical landmark using a deep learning model.

In some kits, the deep learning model comprises a machine learning library, such as one comprising a Pytorch machine learning library, TenserFlow, JAX, KERAS, MXNet, Deeplearning4j, a natural language processing library such as Hugging Face Transformers, OpenCV, NumPY, SciPy, Scikit Learn, or Theqno, among others. In some cases the deep learning library is a Python library, Some deep learning libraries invoke a coding language such as C++. The deep learning model is in some cases validated against at least one annotated reference dataset.

The pose estimation package in some cases evaluates positional information deviation from at least one annotated reference dataset and may display an image comprising the at least one anatomical landmark, record completion of an exercise, and may display completion of the exercise.

The pain alleviation suite receives a first user pain rating, which may be user self-reported. The first user pain rating may be provided prior to the pain alleviation suite generating an output, and may be informed at least in part by an assessment of deviation of user positional information deviation from at least one annotated reference dataset. Some pain alleviation suites recommend a pain treatment, such as a pain treatment comprising mental engagement, such as, generative art creation software, puzzle engagement, music, literature or writing, or other metal activity to direct attention away from a pain source. The generative art creation software in some cases provides a colorable art template, or a visual puzzle.

The pain treatment in some cases comprises a pain medication, alone or in combination with the generative art or other mental activity. The pain treatment is often proposed to the user or to an observing medical professional, such as proposed for approval by a medical professional.

The pain alleviation suite in some cases receives a second user pain rating subsequent to the pain treatment. In some of these cases, the pain alleviation suite assesses effectiveness of the pain treatment, such as in light of the second user pain rating. Pain ratings and assessment are performed, for example, on a computer or device having computing capacity.

The physician portal software package often monitors a user's pain score over time. Additionally or in the alternative, the physician portal software package monitors one or more of a user's change in positional information deviation from at least one annotated reference dataset over time, or a user's physical therapy schedule adherence over time. The physician portal software package in some cases displays a user pain treatment recommendation. So as to facilitate remote monitoring the physician portal software package is accessible through the internet, or on a mobile internet device. The physician portal software package often allows physician user communication. The physician portal software package is implemented, for example, on a computer or device having computing capacity.

Some kits comprise a community interface package, such as one that facilitates performance data exchange between a first user and a second user, such as via wirelessly connected computer interfaces or interfaces among device having computing capacity. The community interface package in some cases comprises program or application directions for implementation on a computer, handheld computing device or cloud implemented computing system.

Disclosed herein are methods of encouraging compliance to a recovery regimen, some such methods comprising one or more of digitally monitoring a user first physical therapy performance, digitally assessing divergence in user first physical therapy performance from a first physical therapy performance model performance, receiving a user first pain assessment, providing a user art based pain management exercise, and receiving a user second pain assessment. Such methods are in some cases implemented, for example, on a computer or device having computing capacity.

The recovery regimen is variously an orthobiologic intervention, a surgical intervention or a physical therapy intervention. Digitally monitoring comprises video recording, and may comprise recording user position monitoring marker location. Data generated thereby is in some cases stored on or transmitted to a computer or device having computing capacity.

Digitally assessing in some cases comprises applying a deep learning model, such as one comprising using a machine learning library, such as a machine learning library that corresponds to a position monitoring output associated with an implantable orthobiologic or other medical composition or material to be applied to a subject delivered to the user. Digitally assessing is in some cases performed on or transmitted to a computer or device having computing capacity.

A number of mental activity or art based pain management approaches are consistent with the disclosure herein, such as drawing or puzzle solving. Alternate mental activity approaches such as word association, vocabulary puzzle, excerpt or text reading, listening to music or similar approaches that distract or focus a user's attention away from physical pain are also consistent with the disclosure herein. Such activities decrease user pain while reducing the risk pain medication addiction risk associated with recovery.

Some aspects of the methods herein comprise comparing the divergence or difference in user first physical therapy performance to the user first pain assessment, alternately or in combination comprising comparing the divergence in user first physical therapy performance to the user second pain assessment, alternately or in combination comprising reporting at least one of the divergence in user first physical therapy performance, first user pain assessment and second user pain assessment to the user. Comparing is in some cases performed on or transmitted to a computer or device having computing capacity.

Reporting variously comprises indicating a value for at least one previous physical performance of at least one of the divergence in user previous physical therapy performance, previous first user pain assessment and previous second user pain assessment to the user or to a third party directing the user. Similarly, in some cases reporting comprises indicating a value for at least five previous physical performances of at least one of the divergence in user previous physical therapy performance, previous first user pain assessment and previous second user pain assessment to the user. The report may comprise an indication of improvement for at least one previous physical performance of at least one of the divergence in user previous physical therapy performance, previous first user pain assessment and previous second user pain assessment to the user. Reporting variously comprises transmission, such as wireless or other transmission, to a computer or device having computing capacity.

Some methods comprise reporting at least one of the divergence in user first physical therapy performance, first user pain assessment and second user pain assessment to a medical practitioner such as a medical practitioner is remote to the user. Alternately or in combination, some methods comprise reporting at least one of the divergence in user first physical therapy performance, first user pain assessment and second user pain assessment to a user community, such as via computer or other computing device, or by posting.

Also disclosed herein are methods comprising providing a movement exercise to a user, generating a record of the user performing the movement exercise, and assessing divergence between the recording and a reference performance of the movement exercise. Some such methods comprise one or more of reporting divergence between the recording and a reference performance of the movement exercise to the user, or reporting divergence between the recording and a reference performance of the movement exercise to a medical practitioner, such as a medical practitioner remote to the user. The movement exercise variously comprises a rehabilitation exercise, such as one targeted to prevent surgical intervention, or to facilitate recovery from surgical intervention, such as an intervention comprising introduction of an orthobiologic or other medical composition or material to, or performance of a procedure on the user or subject. In some cases the intervention is targeted to prevention of harm to the user.

Movement exercises in some cases comprise a surgical rehabilitative, preventative or nonsurgical rehabilitative or preventative exercise, or a nonsurgical exercise such as an athletic training exercise.

Assessing divergence or difference may comprise one or more of performing a computational analysis of the user performing the movement exercise, such as a computational analysis comprising artificial intelligence analysis of the user performing the movement exercise, for example so as to identify one or more of a user body part or parts responsible for the divergence, or user joints responsible for the divergence, or user movement subcomponents responsible for the divergence. Assessing is implemented, for example, on a computer or device having computing capacity.

A reference is in some cases a healthy performance of the movement exercise, or a prior user performance of the movement exercise.

Some aspects of the methods comprise receiving one or more of a self reported pain level from the user prior to performing the movement exercise, or a self reported pain level from the user subsequent to performing the movement exercise.

Some aspects of the methods comprise providing to the user a nonmedicinal pain management exercise subsequent to the user performing the movement exercise, such as one or more of immediately subsequent to the user performing the movement exercise.

Some aspects of the methods comprise receiving a self reported pain level from the user subsequent to performing the nonmedicinal pain management exercise, or prior to performing the movement exercise, receiving a self reported pain level from the user subsequent to performing the movement exercise, providing to the user a nonmedicinal pain management exercise subsequent to the user performing the movement exercise, and comprising receiving a self reported pain level from the user subsequent to performing the nonmedicinal pain management exercise.

Various embodiments of the method comprise generating a report of the assessing, such as generating a report of the assessing and of the patient self reported pain levels from at least one of prior to performing the movement exercise, subsequent to performing the movement exercise and subsequent to performing the nonmedicinal pain management exercise.

A report variously comprises an assessment of whether the user performance constitutes a substantial match to the reference, such as a substantial match comprising at least 80% match to the reference performance.

A report variously comprises one or more of assessment of a degree of divergence from the reference, or extent of compliance to a temporal regimen of movement performance exercises.

The report is provided variously to one or both of the user or to a medical practitioner, such as a medical practitioner remote to the user. The report is delivered, for example, to a computer or device having computing capacity, in some cases via wireless communication.

The method often comprising assessing contents of the report to select a progression course through a movement exercise regimen, such as by selecting a next movement exercise when user performance constitutes a substantial match to the reference. The next movement exercise may be selected from a linear exercise regimen or from among a plurality of immediate or ‘branched’ options, such as may be provided by a medical practitioner. Assessing is in some cases implemented, for example, on a computer or device having computing capacity.

In some cases assessing contents of the report to select a progression course through a movement exercise regimen comprises selecting a next movement exercise when user performance constitutes a substantial match to the reference. In some cases assessing contents of the report to select a progression course through a movement exercise regimen comprises selecting a next movement exercise to target a user joint when user performance differs from the reference related to movement at the user joint. Similarly, assessing contents of the report to select a progression course through a movement exercise regimen may comprise selecting a next movement exercise when at least one user self reported pain level is below a threshold.

Some methods comprise reporting results of the assessing to a medical practitioner such as a medical practitioner is remote to the user, such as via wireless communication.

Also disclosed herein are methods of encouraging compliance to a movement regimen. Some such methods comprise one or more of providing a nonmedicinal pain management exercise to a user promptly or immediately subsequent to user performance of a movement exercise of the exercise regimen, providing a performance assessment to the user promptly or immediately subsequent to user performance of a movement exercise of the exercise regimen, and providing the performance assessment to a medical practitioner, such as a medical practitioner remote to the user.

The user in some cases exhibits improved compliance as indicated by one or more of the following: compliance corresponding to adherence to the exercise regimen better than at least 50% of users provided the exercise regimen without being provided the nonmedicinal pain management exercise immediately subsequent to performance; compliance corresponding to adherence to the exercise regimen better than at least 50% of users provided the exercise regimen without being provided the performance assessment immediately subsequent to performance; compliance corresponding to adherence to the exercise regimen better than at least 50% of users provided the exercise regimen without providing the performance assessment to a medical practitioner.

Also disclosed herein are methods of selecting a physical mobility intervention course, such as methods comprising one or more of digitally monitoring a user first physical performance, digitally assessing divergence in user first physical performance from a first physical performance model performance, receiving a user first pain assessment, and selecting an implantable orthobiologic to introduce into the first user. Digitally monitoring variously comprises one or more of video recording, recording user position monitoring marker location, applying a deep learning model such as one using a machine learning library, for example a machine learning library corresponding to at least one position monitoring output associated with a defect addressable using an implantable orthobiologic or other medical composition or material delivered to the user.

Selecting an implantable orthobiologic, or other medical composition or material, or surgical intervention or other medical course of treatment, variously comprises one or more of weighing user first pain assessment and divergence in user first physical performance from a first physical performance model performance; identifying an implantable orthobiologic, or other medical composition or material, or surgical intervention or other medical course of treatment to address the defect; or assessing user first pain assessment.

Consistent with the above, disclosed herein are kits for practice of any of the methods disclosed above or elsewhere herein.

Consistent with the above, disclosed herein are kits comprising a pose estimation software package, a pain alleviation suite, software package or dataset, and a physician portal software package, each operable on a cloud processor or computer system. Some such kits comprise one or more of a wearable sensor, or an implantable orthobiologic, or other medical composition or material.

Also disclosed herein consistent with the methods and kits above are implantable orthobiologics, such as those comprising one or more of a porous biocompatible matrix, and a population of composite granules. The matrix often comprises a biocompatible flexible material capable of maintaining pores, such as collagen, for example bovine collagen, xenically expressed human collagen, such as from a bacterial or yeast expression system, harvest user collagen, or collagen mimic material. The matrix often comprises a sold structural component, such as dispersed bioactive glass particles. The matrix maintains a porosity, such as one that exhibits a porosity interconnectivity of at least 70%. The matrix maintains a porosity, such as one that exhibits a porosity interconnectivity of at least 80%. The matrix maintains a porosity, such as one that exhibits a porosity interconnectivity of 80%-90%.

Often, the matrix comprises pores of at least 350 um diameter, such as pores of a diameter ranging from 500 um to 600 um, or pores of a diameter ranging from 530 um to 570 um or other sizes consistent with the disclosure herein.

The granules are often embedded in the matrix, in some case so as to convey structural integrity to the matrix. The granules often exhibit a higher density than the matrix. Granules are often heterogeneous, and may comprise one or more of a resorption component and a structural component. The resorption component variously comprises at least 50% of the granule for each granule, or at least 60%, at least 70%, at least 80%, or at least 90% of the granule for each granule. In some cases the resorption component comprises about 95% of the granule for each granule. The resorption component in some cases comprises beta-tricalcium phosphate.

The structural component variously comprises no more than 50% of the granule for each granule, or no more than 40%, 30%, 20%, 10%, or comprises no more than 5% of the granule for each granule, such as about 5% of the granule for each granule. The structural component in some cases comprises hydroxyapatite.

The orthobiologic often comprises an imbibed cell population, such as an imbibed cell population introduced into the matrix. The imbibed cell population may comprise mesenchymal cells. The imbibed cell population may comprise cells from a bone marrow aspirate. The cells are in some cases autologous, or autochthonous. Cells are in some cases harvested from the user prior to insertion of the orthobiologic into the user.

Similarly, some methods comprise harvesting user cells such as mesenchymal cells, bone stem cells, or bone marrow aspirate cells, and infusing said cells into an orthobiologic as disclosed herein or otherwise known in the art, and introduction of the orthobiologic into a user. The orthobiologic is in some cases uniquely or recognizably labeled such that it may be correlated to a treatment regimen or a performance record or performance data set corresponding to user performance.

In addition to orthobiologics, alternat medical compositions or biologics are consistent with the software ecosystem of the disclosure herein. Examples include, for example, regenerative wound dressings comprising bioactive glass compositions as disclosed herein in either particulate or a fibrous patch form to induce post-operative tissue healing. Nonbiologic medical compositions or devices, such as metal splints for bone repair as may occur in compound fracture healing, artificial spinal discs, artificial limbs or other medical intervention devices are also consistent with the software ecosystem or software and non-software kit components herein.

Also disclosed herein are orthobiologics and kits consistent with the methods herein, and methods comprising use of orthobiologics disclosed herein pursuant to practice of the methods herein. Also disclosed herein are software and kits consistent with the methods herein, and methods comprising use of software for computer implementation to practice the disclosure herein pursuant to practice of the methods herein.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

FIG. 1 shows an orthobiologic material withstanding stress of being twisted.

FIG. 2 shows a user performing a prescribed physical therapy exercise while a convolutional neural network extrapolates the user's joint positions pursuant to assessing proper exercise performance.

FIG. 3 shows a mobile device presenting physical therapy adherence and accuracy of performance over time.

FIG. 4 shows a medical practitioner accessing a patient's physical therapy and art therapy adherence from a remote portal.

FIG. 5 shows a schematic of a kit disclosed herein, comprising an implantable orthobiologic, pose estimation software package, pain alleviation suite and physician portal software package.

FIG. 6 shows a system and workflow disclosed herein, comprising a video capture device that captures images of an individual wearing wearable motion sensors, and that communicates the data to a computer such as a cloud for data analysis to produce a three dimensional pose estimation that may be compared to a reference.

FIG. 7 shows an identification code and associated information used in various embodiments.

FIG. 8 shows a pathway for assessing data to produce pose progression or pain amelioration progression performed on a portable computing device (shown) or in a computing cloud.

### DETAILED DESCRIPTION

Disclosed herein are compositions, systems and methods for user mobility assessment, such as may be used in combination with any one or more of orthobiologic or other surgical or other medical intervention, surgical or surgical alternative assessment, or traditional or alternative pain management. Some such systems comprise digitally-enabled orthobiologics or other medical composition or material platforms that feature one or more of a mobile application, Artificial Intelligence-driven or other software, a desktop-based or other digital portal, and an implanted or injected orthobiologic or other medical composition or material linked to the patient rehabilitation software through a unique or other activation code, and which may select or provide traditional or alternative pain recommendations.

Systems and methods are discussed herein in distinct sections, but it is understood that compositions and systems are contemplated as being used to perform methods disclosed herein as well as other methods, while methods disclosed herein may comprise use of disclosed systems or other compatible systems. Accordingly, it is understood that disclosure from any section may be understood in the context not only of that section but of the disclosure generally.

### Orthobiologic and Other Systems for Physical Therapy

Disclosed herein are orthobiologic compositions for surgical intervention, and systems for recovery from surgery such as orthobiologic or other medical device or composition introduction into a user. In cases comprising orthobiologic or other surgical intervention, after implantation or injection of the orthobiologic or other medical composition or material, or performance of the surgical procedure, a treating or other healthcare provider can enroll the patient or user into a digital recovery platform using a unique or specific identification code associated with the particular orthobiologic, other material, or procedure. Alternately a user may self-enroll or be enrolled by other third party. Once enrolled, the recovery profile of the treated patient or user is populated with the lot number and the biochemical characteristics of the orthobiologic or other material used, or features of the intervention, which may be correlated to an identifier of the orthobiologic or other medical composition or material, such that the kit identifier or orthobiologic or other medical composition or material identifier or identification code is associated with features comprising one or more of a recovery profile of a user or recipient, a user or patient insurance status or billing code, biochemical or other characteristics of the orthobiologic or other medical composition or material, and diagnosis of the user or recipient, alone or in combination with other information. Alternately, such as in cases of surgical selection or surgical alternative selection, or other use of the technology disclosed herein, the user or a medical practitioner, for example, may enter details of a diagnosis or other identifier into the digital platform.

The digital platforms disclosed herein are often customizable to each or a particular patient, recipient or user. Accordingly, unique physical therapy and pain management plans can be prescribed and monitored by a treating healthcare provider and correlated or directed to a patient or user. An exemplary method of delivery to a patient or user is through a mobile application that leverages the patient's on-device camera to track their kinematic motion, such that collected data may be used to assess whether the prescribed physical therapy exercises have been sufficiently completed.

Additionally, the mobile application in some cases allows a patient or user to engage in alternate pain therapy such as art therapy or mental activity therapy for pain management, for example using AI-generated or previously generated literary or visual artwork for on-device coloring or puzzle completion, as well as in some cases having the ability to select accompanying sounds from a library of pre-installed or accessed recordings. The patient or user is in some cases prompted to enter their pain score before, after, or before and after each art therapy session or other mental activity session. In various embodiments, a treating healthcare provider can use the desktop-based portal to monitor the patient's or user's physical therapy adherence and pain score progress as well as examine granular data on exercise repetitions and the effectiveness of specific art therapy or other mental activity sessions. Working in concert to inform the future treatment plans of the patient by the healthcare provider, one or more of the combined orthobiologic data, AI-enabled physical therapy analysis, and AI-generated art therapy pain management progress are interpolated by the technology to create a unique or user personalized bio-digital patient recovery profile.

Through use of software such as may direct a computing system implementing some embodiments of the mobile application, the patient is able to send and receive one or more of asynchronous text, image-based, and video messages to the healthcare provider or to additional users in an open or closed user community. A user is in some cases able to schedule a rehabilitation session, art session, other mental activity session or art session and rehabilitation session so as to complete said session or sessions in concert with fellow users, either in physical proximity or remove from one another, using interfaces provided by the application. Concurrent scheduling in some cases comprises sending an invitation to an individual or group, and upon acceptance of the invitation, establishing a joint session either in physical proximity or through an interface to facilitate one or more of remote communication and result sharing. Concurrent session performance in some cases increases the chance or frequency of session completion by at least, at most, about or exactly 10%, 20%, 30%, 40%, 50%, 75%, 100% or more.

Additionally, the desktop-based portal, as may be directed by software or an application of a kit herein, or may be a part of a kit herein, allows the healthcare provider or other user to continuously enter additional patient data derived from external sources such as, for example, one or more of comorbidity and body mass index data as well as self-reported assessments of the patient's diagnostic imaging taken during the course of recovery.

Compositions, systems and methods disclosed herein are further understood in light of the following discussion of particular system component, which may in some cases correspond to or enable particular method steps or may be constituents of kits or platforms upon which software or applications of kits may act.

Orthobiologic Component: An implantable or injectable orthobiologic or other medical device or composition is a component of some systems and methods herein, such as for bone regeneration in the spine, skeletal extremities, the pelvis, or any bone suffering from traumatic injury. Orthobiologics may be engineered from a broad range of biocompatible or bioactive components, such as bioactive glass, beta-tricalcium phosphate, hydroxyapatite, and collagen such as animal sourced collage such as bovine collagen or expressed transgenic collagen as may be produced from a bacterial or microbial system.

Orthobiologics include varieties with osteogenic, osteoinductive, and osteoconductive capabilities. These varieties include allografts, xenografts, and wholly synthetic biomaterials. Allografts can be defined as bone transplanted from a donor to a patient and are typically harvested from either a cadaver or a living donor. Xenografts can be defined as animal-derived bone with similar properties to allograft. Synthetic biomaterials are engineered, bone-mimicking or osteogenic materials that include ceramics, biopolymers, and peptides. Allografts, Xenografts, and the majority of synthetic biomaterials are delivered intraoperatively with certain injectable orthobiologics delivered in a clinical setting.

In exemplary embodiments, an orthobiologic mimics the patient's own bone to allow for healing in defects without the need to harvest tissue such as bone from another site in the body. Alternate orthobiologics are autologous or comprise material drawn from a patient's body, or are autochthonous, xenically grown or synthesized, or drawn from a third party individual or entity.

Features common to many orthobiologics include being formed from a porous matrix. Porosity in some cases allows user cell invasion prior to, or subsequent to, or both prior to and subsequent to surgical implantation. Matrix porosity is often of pore sizes comparable to or greater than user cells in the vicinity of the orthobiologic insertion site.

Exemplary orthobiologics exhibit an interconnected porosity of about, at least or at most 50%, 60%, 70%, 75%, 80%, 85%, 90%, or 95%, or a range spanning or outside of the values listed therein. Exemplary embodiments exhibit an interconnected porosity ranging from about or exactly 50% to 95%, 60% to 95%, 70% to 95%, 80% to 90%, 83% to 87%, about 85% or 85%.

A broad range of pore sizes are consistent with the disclosure herein. Pore sizes are in some cases selected to facilitate uptake of imbibed cells such as imbibed mesenchymal stem cells as may be harvested from a user or a third party. Mesenchymal or other cell populations may be obtained by any of a broad range of approaches know in the art. Similarly, pores are also selected to facilitate nutrient and oxygen transport to the osteoblasts after attachment.

Pores consistent with the disclosure herein exhibit a mean or median diameter of about, at least or at most 100 um, 150 um, 200 um, 250 um, 300 um, 350 um, 400 um, 450 um, 500 um, 550 um, 600 um, 650 um, 700 um, 750 um, 800 um, 850 um, or 900 um, or a range spanning values listed therein. Exemplary embodiments exhibit a mean or median pore size ranging from 350 um-750 um, 400 um-700 um, 450-650 um, 500 um-600 um, 530 um-570 um, 540-560 um, about 550 um to 550 um.

So as to facilitate cell invasion, some orthobiologics are imbibed with user or third party cells to encourage cell invasion of the orthobiologic, prior to or concurrent with application to a user. A suitable cell population for such pre-surgical cell invasion is mesenchymal cells, or cells as may be obtained from a bone marrow aspirate, or other cells conducive to invasion of the orthobiologic matrix.

A broad range of biocompatible compositions are suitable matrix constituents. Exemplary matrices comprise collagen, though other biocompatible structural components compatible with porous matrix formation, cell invasion and resilient to the stress of introduction into a user are also contemplated. Some discussion of collagen and biomaterial scaffold formation is found in Drury and Mooney (2003) “Hydrogels for tissue engineering: scaffold design variables and applications” Biomaterials 24:4337-4351, which is hereby incorporated by reference in its entirety.

Matrices are in some cases supplemented or fabricated using granules. Addition of granules to a matrix facilitates structural integrity, so as to allow larger or more frequent pores without surrendering structural integrity. Granule embedded matrices allow imbibing of a greater proportion of mesenchymal stem cells or other cells, and is conducive to imbibed cell survival due to maintaining structural integrity. Similarly, granule embedded matrices facilitate dispersion of stability factors such as bioactive glass through the matrix.

A broad range of granules are consistent with the disclosure herein. Granules denser than the matrix in which they are embedded are preferred in many embodiments, such as denser than collagen or denser than collagen and bioactive glass. Exemplary granules are selected to exhibit a high resorption rate, such that they are in some cases gradually broken down in the body of a user, so as in some cases to be resorbed or released into the circulatory system. Alternately or in combination, exemplary granules are selected to exhibit a high structural stability prior to resorption, so as to allow long term stability of the orthobiologic and facilitating its replacement with native user tissue such as native user collage as may be involved in supplementation of replacement of the orthobiologic with native user bone or other tissue.

Accordingly, granules variously convey a structural functionality and a resorption functionality, either in a single composition or as a mixture of constituents. Some exemplary granule compositions comprise a high resorption constituent such as beta-tricalcium phosphate and a structural constituent such as hydroxyapatite. Various proportions of these high resorption constituents and structural constituents are consistent with the disclosure herein, such as 50%/50%, 60%/40%, 70%/30%, 80%/20%, 90%/10%, 95%/5%, 99%/1% or other proportions within, spanned by or outside of the range of proportions presented herein. Some exemplary granules comprise a ratio of 19:1% beta-tricalcium phosphate to hydroxyapatite, such as 95% beta-tricalcium phosphate and 5% hydroxyapatite.

Similarly, some granules comprise beta-tricalcium phosphate and hydroxyapatite in molar proportions about, at least, at most or exactly 1:1, 2:1, 5:1, 10:1, 20:1, 30:1, 40:1, 50:1, 55:1, 60:1, 61:1, 62:1, 63:1, 64:1, 65:1, or 70:1, or a proportion spanned by our outside of the range of the ratios given herein.

Proportions as those listed herein convey particular benefits over the art. For example, prior art granules exhibiting a ratio of less than 1:1 (that is proportionally more hydroxyapatite than resorption agent) do not facilitate resorption or granule replacement by user cells, bone or collagen. Similarly, prior art granules having resorption components but lacking structural components often suffer from structural defects such that they may lose structural integrity pursuant to imbibing with cells or subsequent to introduction to a user, prior to calcification or replacement with user bone to maintain structural integrity.

In addition to orthobiologics, alternate medical compositions or biologics of similar composition or standard medical compositions are consistent with the software ecosystem of the disclosure herein. Examples include, for example, regenerative wound dressings comprising bioactive glass compositions, as disclosed herein in either particulate or a fibrous patch form to induce post-operative tissue healing. These biologic compositions share composition characteristics of the orthobiologics as discussed above. Nonbiologic medical compositions or devices, such as metal splints for bone repair as may occur in compound fracture healing, artificial spinal discs, artificial limbs or other medical intervention devices are also consistent with the software ecosystem or software and non-software kit components herein.

An implant or alternative medical composition or construct is in some cases delivered in hermetically-sealed packaging with a unique or distinguishing identification code, which may be printed on the package and on one or more, such as 2, 3, 4, 5, 6, 7, 8, 9, 10 or more than 10 removable stickers. Alternately, or in combination an implant may harbor an internal or removable chip that may provide or broadcast the identifying code.

An identifier or identifying code may convey a broad range of information. Some identifiers or identifying codes identify the orthobiologic or other alternative medical composition or construct. As discussed elsewhere herein, an identifying code may further be associated with one or more of the following: biochemical characteristics of the orthobiologic or other alternative medical composition or construct, a user or recipient of the orthobiologic or other alternative medical composition or construct, a user or recipient condition to be addressed by the orthobiologic or other alternative medical composition or construct, a user or recipient diagnosis, a user or recipient exercise or movement dataset, as may be captured prior to a surgical intervention, a user or recipient recovery profile, a target exercise dataset, or other information related to either the orthobiologic or other alternative medical composition or construct (such as orthobiologic identity, chemical composition or structure), or to the user or recipient, task performance either prior to, subsequent to (after it is generated) or independent of the user. In some cases the identifier or identification code is initially associated with the orthobiologic or other alternative medical composition or construct, and is subsequently associated with data related to the user or recipient, such as performance data, diagnosis, self-reported pain profile data, medical professional diagnosis or progression recommendation, or other user or recipient information. That is, an identification code may initially be assigned only to an orthobiologic, but upon selection of the orthobiologic for a user intervention, the identification code may be associated with the user, user condition, user diagnosis, and a reference pose dataset, for example. Pursuant to rehabilitation or recovery, user rehabilitation pose data, deviation of user pose data from a reference, user reported pain levels, such as one or more of prior to or subsequent to performance of a rehabilitation exercise or prior to or subsequent to performance of a pain alleviation task or exercise, user recovery profile, medical professional assessment, recovery progression status or other information related to the user may be associated with the identification code.

Subsequent to unsealing an implant or orthobiologic, the orthobiologic is in some cases imbibed with cells, such as mesenchymal stem cells, for example from bone marrow aspirate. As mentioned above, the orthobiologics as disclosed herein exhibit greater structural stability, facilitating greater porosity and greater cell recruitment subsequent to imbibing. These improved features arise in part from the introduction of granules such as granules comprising 95% beta-tricalcium phosphate and 5% hydroxyapatite, or other proportion disclosed herein, and in some cases bioactivated glass. Consequently, orthobiologic compositions herein recruit more cells that inserts in the art. That is, some such compositions exhibit in some case a cell recruitment rate of at least, at most, about or exactly 10%, 20%, 30%, 40%, 50%, 75%, 100% or more greater than orthobiologics in the art.

The packaging is designed to allow a physician's team to either scan or physically remove one of the corresponding stickers to facilitate the subsequent enrollment of the patient into a system herein, such as a digital physical rehabilitation and art therapy platform. When the sticker is scanned using a mobile application, or an identifier is otherwise inputted, the physician is prompted to enter one or more of the patient's or user's healthcare details and email address. The patient may be sent a direct link to download the corresponding, patient-facing mobile application. Once the patient downloads the app using the email invitation, their physical rehabilitation and art therapy progress will be viewable by their own healthcare provider. The unique or identifying ID creates a direct link between the specific implant that was used and the subsequent digital recovery journey

A number of orthobiologic alternatives are consistent with the disclosure herein. Generally, any medical device, tool or material is consistent with some embodiments herein. That is, some kits comprise, rather than an orthobiologic, a plate or plates to stabilize or resolve a compound fracture, an artificial organ or tissue, a tissue transplant, a graft or cadaver tissue such as an Anterior Cruciate Ligament or other ligament, a cultured tissue, an artificial heart valve or stent, sutures or other material used in a surgical procedure. A common feature of some such medical device, tool or material embodiments is that they are assigned or associate with an identifier or identification code as described herein, so as to associate the medical device, tool or material with one or more of a recipient, a condition, a rehabilitation exercise model dataset, a user dataset upon its generation subsequent to an intervention, a pain status, or other data as may be relevant to user or recipient recovery monitoring.

Pose-Estimation Software Component: Some systems comprise pose-estimation software, such as a pose estimation software package operable on a cloud processor or computer system. Pose estimation software directs or has the capacity to direct a computer or device having computing capacity, such as a smart phone, to perform pose estimation functions consistent with the disclosure herein. Software may include a convolutional neural network trained with the ability to suggest proper, corresponding insurance billing codes for the application of the bioactive glass particulates or the bioactive glass fibrous patch by interpreting the dictated description of the procedure. Pose estimation software may allow for the visual capture, annotation, depth and area estimation, infection prediction, and healing assessment of the orthobiologic delivery site and any corresponding wound using convolutional neural networks trained on a wound database. Some such software leverages the depth-sensing camera on a user's mobile device to capture and compare a video feed, for example of the real-time movement data of a patient during a physical rehabilitation session with kinematic models for each of the pre-loaded exercises. Alternately or in combination, some pose estimation software incorporates or collects positional marker data such as that generated from positional markers attached to a patient or user, or directs a computer or image capture device that in some cases bas computational capacity, such as a smart phone.

The pose-estimation software in some cases automatically undergoes preprocessing to filter noise before the software applies computer vision algorithms to analyze the video frames of the patient, such as in real time, to assess user or patient pose. Alternatively in some cases processing occurs after the software applies computer vision algorithms to analyze the video frames of the patient, such as in real time, to assess user or patient pose, or in some cases no processing occurs. Pose estimation in some cases comprises identification of key anatomical landmarks such as joints.

Some such software operates using an a priori algorithm, while alternatives are trained on datasets, such as large annotated datasets. Exemplary software embodiments detect a patient's joints through the use of deep learning models developed using a deep learning library such as the Pytorch machine learning library, TenserFlow, JAX, KERAS, MXNet, Deeplearning4j, a natural language processing library such as Hugging Face Transformers, OpenCV, NumPY, SciPy, Scikit Learn, or Theqno, among others. In some cases the deep learning library is a Python library. Some deep learning libraries invoke a coding language such as C++.

One exemplary deep learning model consistent with the disclosure is a Convolutional Neural Network or other artificial neural network for analysis of visual imagery. Exemplary Convolution Neural Networks comprise one or more of the following steps, which may also be adopted by alternate deep learning or AI models.

The Network applies a filter to the visual exercise data obtained from the patient and produces a feature map that highlights regions of the input that match the filter's pattern.

Some exemplary Convolutional Neural Networks or other software suitable for processing image or grid-like data operable on a computer employ one or more of several layers of increasing specificity to identify the patient's joint positions. The first layer is the input layer, which is the raw sequence of video frames from the patient's exercise recording that undergoes instant preprocessing for performance. The next layer is the convolutional layer which extracts low-level features such as edges and textures. The following layer is the pooling layer, which reduces spatial dimensions for computational efficiency while retaining essential image information. The subsequent layer is the heatmap layer, which is a probability-driven output that features a set of heatmaps, one for each joint. The highest intensity points in these heatmaps correspond to the predicted joint locations. This heatmap is a two-dimensional representation where the intensity at each pixel indicates the probability of a patient's joint being located at that position. The final step in the Convolutional Neural Network is post-processing, which identifies the peaks in heatmaps to identify the patient's joint positions during exercises.

In an exemplary CNN approach, 3D pose estimation and comparison to a reference is effected through one or more of the following steps. An individual wearing motion sensors performs a pose exercise regimen under the view of a video capture device such as a phone. The video images are transmitted to a cloud computing locale where they are subjected to analysis by a convolutional neural network, comprising 2D joint estimation based upon wearable motion sensor data alone, user image data alone, or a combination of the two. Joint probability is then mapped based upon the 2D estimation. The mapped joint probability data is subjected to depth triangulation or other analysis to generate three dimensional joint probability data that may be used as a three dimensional depiction or representation of a user pose which can readily be compared to a reference joint dataset, such that difference from or similarity to a reference can be calculated.

The proper prediction of the patient's joints is made possible in some cases by iteratively training the Convolutional Neural Network on large scale musculoskeletal datasets, enabling the application to use joint recognition to offer corrective guidance in relation to the prescribed physical therapy exercise.

Alternately or in combination, joints or other patient regions are identified using position specific labels attached to a patient or user, or broadcast signals transmitted from an orthobiologic insert.

Some deep learning models are validated by singularly or iteratively comparing and optimizing or improving their accuracy in predicting anatomical landmarks against annotated reference data. As a user performs each prescribed exercise, she or he is in some cases able to see his or her own kinematic model with anatomical landmarks on a screen such as a mobile device screen. In some cases, a user or patient is rewarded with, for example, one or more digital badges or other indicia of progress upon completion of a prescribed repetition or exercise. Daily physical rehabilitation exercise adherence may be recorded and available for review by a third party such as a co-rehabilitating individual or group, or a treating healthcare provider, such as through their portal.

Some deep learning models effect visual capture and depth area estimation so as to generate user posture data models for comparison not a reference. Models are in some cases trained on a wound model database, so as to facilitate prediction of healing progression or healing assessment, infection prediction or other assessment of an orthobiologic delivery and integration into a user, or other medical device or composition evaluation.

Pain Alleviation Software Component: Some systems comprise a pain alleviation component such as an art therapy, mental puzzle, foreign language quiz, math puzzle, literature or other intellectual exercise to provide or recommend a pain alleviation approach. Some pain alleviation approaches comprise recommending conventional pain medications, which may be administered by the patient or user or may be prescribed by a treating healthcare provider, such as through a practitioner portal.

Alternately or in combination, some systems comprise generation de novo or customization and provision of third-party generative art creation software, or provision of third party art. Some such software allows a user or patient to use their mobile device to perform a mental activity, such as coloring in or solving a visual puzzle, as may be derived from previously existent or newly created digital artwork such as may be generated daily. Before the user begins the art therapy session, they are in some cases asked to select from a collection of nonpharmaceutical pain alleviation approaches, such as whether they would prefer to color, solve a visual puzzle, work on a mental exercise, practice a foreign language quiz or other mental exercise. Once they have made their selection, or prior to their selection, they are in some cases prompted to enter their current pain score, for example on a scale of 0 to 10 or other self-assessed or externally evaluated approach for pain assessment. Alternately, or in combination, a user or patient is asked if they would like to select music to play from the application's library of public domain or proprietary music during the session. Concurrent with or after completion of a nonpharmaceutical pain alleviation regimen, they are prompted to enter their current pain score, which is entered and may be provided on a physician's portal or other practitioner portal. In some cases, the total time of the art therapy session is recorded alongside the two pain scores in their recovery profile. Various embodiments of art therapies or other alternate pain reduction therapies exhibit a pain reduction of at least, about, or no more than 5%, 10%, 15%, 20%, 25%, 30%, 35%, 40%, 45%, 50%, 55%, 60%, 65%, 70%, 75% 80%, 85%, 90%, 90%, or up to 100%.

Pose-estimation and Pain Alleviation Component Synergy. Software components in some cases allow for a synergistic intertwining of data collected and processed from the Pose-Estimation and Pain Alleviation or other Art Therapy components into a composite assessment. A composite assessment may allow a user or medical practitioner to extrapolate insights that consider both physical therapy adherence and Art Therapy adherence for individual pain improvement. The composite assessment may enable the software application to recommend alternative Art Therapy pathways, such as in the case that physical therapy adherence has been confirmed by the software without an improvement in the pain score over a set period, such as over a number of sessions or a period of time, for example, 1, 2, 3, 4 5, 6, or 7 or more days, or 1, 2, 3, 4 or more than 4 weeks, 1, 2 or more than 2 months, for example over two week intervals. Users are in some cases also provided with a comparative score of their Physical Therapy adherence in relation to their Pain Alleviation such as Art Therapy adherence.

Pose estimation and Pain Alleviation in some cases synergize in the assessment of progression decisions for a rehabilitation course. That is, in some cases progression is informed by comparison of user pose capture data to a reference dataset, such that deviation by no more than a certain amount from a reference ‘improved’ or ‘healthy’ or ‘target’ dataset leads to a progression decision, such as deviation by no more than 1%, 2%, 5%, 10%, 20%, 50% or greater, or a number spanned by or outside of this range, leads to a decision to progress. Alternatively deviation by at least a certain amount from a reference ‘baseline’ or ‘prior to intervention’ or ‘starting’ reference dataset leads to a progress decision, such as deviation by at least 1%, 2%, 5%, 10%, 20%, 50% or greater, or a number spanned by or outside of this range, leads to a decision to progress.

In some cases, progression decisions are informed not merely by deviation from a reference but also by pain status, such as user reported pain levels. That is, progression decisions are based upon not only deviation from a reference, such as a baseline pose or a target pose, but also by patient reported pain levels. These pain levels may be reported in the context of pose exercise performance, such as immediately after pose exercise performance or pose capture or at some longer time after pose exercise performance. Pain level reporting may serve as a separate gate to progression, such that self-reported pain levels above a threshold impede progression through a rehabilitation course independent of deviation from a reference, such as a baseline pose or a target pose. Similarly, in some cases reported pain levels may be combined with reference deviation data so as to generate a combinatorial metric that is used to make progression decisions. That is, percentage deviation from a baseline or reference may be multiplied by percentage deviation from a pain-free report to generate a progression metric, that is itself assessed relative to a threshold value to inform progression decisions. This is one example of a larger general scenario where both deviation from a reference and deviation from a pan free report or from a baseline pain level are used in making progression decisions. Various alternate mathematical equations, learning models or algorithms may be used to come to a progression decision, or in some cases the pose reference and pain reporting are independently evaluated against thresholds and progression decisions are made only when both values are above a threshold or otherwise indicative of progression. More complex approaches, where one or the other metric is weighted relative to the other, one or both metrics are subjected to manipulation such as squaring or other exponent manipulation, are also contemplated, as are approaches where localized pain reports are assessed in the context of localized pose deviation, such that assessments are not based upon aggregate pain reporting or deviation or aggregate pose estimation deviation, but on one or more individual joints or portions of a pose and pain localized thereto or distal therefrom.

In some cases, progression decisions are informed not merely by deviation from a reference but also by pain status response to pain alleviation therapy. That is, alone or in combination with any of the above, progression decisions are informed not merely by one or more of pose percentage deviation from a baseline or reference and pain self-reporting levels, but by pain self-reporting level amelioration resulting from pain alleviation task completion. That is, as an alternative to or in addition to pain level reporting, one may assess the degree to which pain persists or is responsive to pain alleviation in making progression decisions. For example, one may reduce the proportionate impact of a high self reported pain score associated with a pose or exercise if that pain score is highly responsive to pain alleviation task completion, while emphasizing or not reducing the proportionate impact of a high self reported pain score associated with a pose or exercise if that pain score is poorly responsive or unresponsive to pain alleviation task completion. Similarly, one may use self reported pain scores after alleviation exercise completion rather than pain scores prior to alleviation exercise completion in making pose progression decisions.

Any or all of the data captured or used in progression decisions is in some cases associated with an orthobiologic or other medical device or composition identification code, such that the identification code may serve as a tag for a broad variety of data associated with use of the kits or practice of the methods herein.

Physician Portal Software Component: A physician's or practitioner's portal facilitates communication, evaluation of or oversight of a user's or patient's rehabilitation or therapeutic regimen. Various portals allow a practitioner to monitor and chart the patient's daily pain score, physical rehabilitation, and art therapy adherence, add additional data such as comorbidities and body mass index scores. The portal is accessible, such as through the web and is often navigable on a desktop or mobile device. The portal also gives a practitioner the ability to view which CPT (Current Procedural Terminology) codes they are eligible to be reimbursed for by monitoring the patient's remote physical rehabilitation, pain score progress, and art therapy sessions. The portal may also include an option to send an asynchronous text or video message to the patient's inbox or to call them on their phone number. Similar portals may share user regimen progress to a nonphysician third party, for example a co-rehabilitating individual or group, as may be beneficial to reenforce or encourage compliance through peer pressure, competition, or peer reinforcement.

Sensor components. Some kits further comprise sensor components, such as components indicative of user posture, position, movement, joint or other body part location. Some sensors indicate orthobiologic or other medical component or intervention site location. Sensors may be attached to the user, worn by the user, for example at joints or positions indicative of joint location, or integrated into a carried or worn user device such as a phone or watch. Some systems employ a plurality of sensors, such as in joint proximity, while other systems rely upon a single sensor such as a watch or carried phone. Alternate systems derive joint or user posture information from an image, images, or a recording of patient movement pursuant to executing a regimen constituent movement or movements, for example using pose identification software as described herein. A sensor components may be attached to a stretchable band or other material, to non-stretchable adjustable band or belt, applied, such as via an adhesive directly to the user, or attached directly to user clothing that is worn pursuant to pose image capture to facilitate joint or other datapoint identification.

A sensor may actively broadcast a signal that is received by an image capture device, or may passively mark a user position such that that position is more readily identified in a user image capture dataset. Alternatively, some image capture and image capture data evaluation approaches identify user body positions without the use of an exogenous sensor component, instead relying upon, for example, user image data to identify relevant user positions of, for example, joints or body parts. In these cases the kits may not comprise specific sensor components to be attached to a user, though they may comprise a computational capacity such as a computer or cloud implemented software component to detect user position.

### Movement-Pain Evaluation

System components such as those above facilitate practice of a broad range of methods relating to movement evaluation, alone or in combination with pain alleviation through medicinal or nonmedicinal approaches.

Evaluating mobility and addressing mobility associated pain. A common theme of a plurality of such methods relates to mobility evaluation, often in combination with addressing mobility associated pain. This pain may arise from orthobiologic or other medical product or composition administration to a user, or may be a symptom which orthobiologic or other medical product or composition administration to a user is intended to address. Various embodiments relate to applying such methods to evaluation of mobility impairment and associated pain in the context of patient evaluation pursuant to surgery selection, identification of a joint or region for a surgical intervention such as an intervention comprising delivery of employment of an orthobiologic component or other medical material, or for a nonsurgical intervention regimen.

Alternately, some embodiments relate to mobility performance rather than mobility impairment, as in the case of practice of the methods and use of systems herein by for example an athlete to assess performance of an athletic task or a performer to assess execution of a particular maneuver such as a dance maneuver. In some such cases, pain evaluation is replaced by difficulty assessment or fatigue assessment.

Practice of these methods variously comprises one or more of capturing motion information from a user, tagging the information such that it may be stored for temporal comparison or correlated with the user or patient, in particular the intervention or orthobiologic or other medical material introduced into the patient or user. Motion may be captured through use of a video recording device such as a handheld mobile device, through use of a carried or attached movement detector, alone or in combination with position or joint indicative markers that may be worn or attached to positions on a user or patient.

A motion capture video, such as generated by a depth sense camera, or dataset is compared to a reference so as to evaluate mobility performance. The performance is variously performance of a routine task such as walking or an untargeted measurement of motion throughout a period of time. Alternately, in some cases data is captured pursuant to patient performance of one or more physical therapy or mobility evaluation exercises or activities, such as physical therapy or mobility exercise or activities prescribed either for mobility evaluation or for mobility improvement, such as pursuant to physical therapy, or pursuant to performance of an athletic task or a performance maneuver such as a dance maneuver.

A number of references are consistent with various embodiments of the technology herein. Exemplary references include a dataset generated from at least one prior performance of the evaluation exercise or activity by the user or patient, such as before an intervention or subsequent to an intervention, or an idealized or healthy performance of the evaluation exercise or activity.

Some methods comprise data preprocessing to filter noise before the software applies computer vision algorithms to analyze the video frames of the patient, such as in real time, to assess user or patient pose. Pose estimation in some cases comprises identification of key anatomical landmarks such as joints.

Some methods comprise using an a priori algorithm, while alternatives are trained on datasets, such as large, annotated datasets. Exemplary methods detect a patient's joints through the use of deep learning models developed using a deep learning library such as the Pytorch machine learning library, TenserFlow, JAX, KERAS, MXNet, Deeplearning4j, a natural language processing library such as Hugging Face Transformers, OpenCV, NumPY, SciPy, Scikit Learn, or Theqno, among others. In some cases the deep learning library is a Python library, Some deep learning libraries invoke a coding language such as C++. Alternately or in combination, joints or other patient regions are identified using position specific labels attached to a patient or user, or broadcast signals transmitted from an orthobiologic insert. Some deep learning models are validated by singularly or iteratively comparing and optimizing or improving their accuracy in predicting anatomical landmarks against annotated reference data. As a user performs each prescribed exercise, she or he is able to see his or her own kinematic model with anatomical landmarks on their mobile device screen.

In some cases, a user or patient is rewarded with one or more digital badges or other indicators of task completion upon completion of a prescribed repetition or exercise. Daily or other regular or target physical rehabilitation exercise adherence may be recorded and available for review by a treating healthcare provider, such as through their portal, or to one or more peer rehabilitating individuals, as may form a peer group.

Mobility evaluation and pain assessment are in some cases effected through use of the kit components described elsewhere herein, such that disclosure in those sections is informative of some embodiments of methods of performing these tasks.

Rehabilitation progression gating. Some methods further relate to prescribing a gated change in a rehabilitation regimen task in response to completion of a prescribed repetition or exercise. Completion is assessed in light of performance of a particular exercise task, performance of a particular exercise task at above a threshold of similarity to or below a threshold of variation from a reference such as an a priori reference or other reference contemplated herein or elsewhere, performance of a particular exercise task at a self-reported pain level below a threshold, or both above a threshold of similarity to or below a threshold of variation from a reference and below a self-reported pain level threshold, the pain level threshold being reported at one or more of before performance of the exercise, after performance of the exercise, and after performance of a pain alleviation task.

Gated changes in a rehabilitation regimen often comprises providing a change in rehabilitation regimen or progression through a rehabilitation regimen in response to evaluated performance. The regimen is in some cases selected by a medical practitioner, and progression through gated steps in the regimen is often confirmed or approved by a medical practitioner prior to being provided to the user. Alternatively, some regimens are associated with an identifier or identification code of an orthobiologic or other material associated with or used in a surgical intervention, or with the surgical intervention itself. A benefit of some of the systems and methods herein is that a preliminary or dispositive assessment of user performance is provided to the medical professional, such that the medical professional does not need to observe individual performance of particular exercises by the user. Rather, the systems or methods provide a performance assessment to the medical practitioner, in some cases accompanied by video or still images relevant to or informative of performance, such that the medical practitioner may assess performance, alone or in combination with patient pain self-assessment.

Progression is in some cases linear, in that a user either progresses or does not progress from a first exercise to a second exercise in light of an exercise evaluation, alone or in combination with a self-reported pain assessment. The exercise evaluation is performed either by a system herein or by a medical professional to which performance images, performance results or performance images and results, alone or in combination and pain self-reporting is reported or images or video information provided.

Alternately, progression is in some cases bifurcated or branched, such that a second exercise is selected from a plurality of second exercise alternatives in light of an exercise evaluation, alone or in combination with a self-reported pain assessment. The exercise evaluation is performed either by a system herein or by a medical professional to which performance results or performance results and pain self-reporting is reported or images or video information provided.

Self-reported pain assessment is evaluated in some cases in light of reported pain level prior to exercise performance, reported level subsequent to exercise performance, reported level subsequent to performance of a system-provided alternative pain amelioration activity such as art therapy, for example a mental activity such as coloring in or solving a visual puzzle, or even degree of change in self-reported pain level between any of the reporting checkpoints mentioned above such as prior to exercise, subsequent to exercise or subsequent to a pain amelioration activity. That is, exercise regimen progression may be gated by any one or more of system evaluation or medical practitioner evaluation of exercise performance, self-reported pain level, or change in self-reported pain level pursuant to an exercise execution.

Progression may comprise selecting or proposing, autonomously or resulting from providing results to a medical professional, escalation of difficulty or number of repetitions of an exercise, progression to a second exercise, ceasing or reducing difficulty of an exercise, isolating or removing a particular component or movement in an exercise, alternating among exercises or between a first exercise and a second exercise, or completion of an exercise regimen. Exercise progression may comprise proceeding through a set of exercises focusing on a particular orthobiologic component or intervention relating to a particular orthobiologic component, alone or in combination with orthobiologic component-independent exercises. An exercise regimen is in some cases associated with an orthobiologic or other medicinal component identifier or identification code.

Like other methods herein, rehabilitation gating is in some cases effected through use of the kit components described elsewhere herein, such that disclosure in those sections is informative of some embodiments of methods of performing these tasks.

Compliance incentivization. Systems and methods herein may in some cases track performance of an exercise or regimen, or a progression among a plurality of exercise in an exercise or rehabilitation regimen. Performance is in some cases tracked as completion of a first exercise in an exercise regimen. Alternately or in combination, performance is evaluated as degree of compliance to or conformation to a target or model reference data set such as a set contemplated herein.

Progression is in some cases reported to a medical professional. Alternately or in combination, performance is reported to the user or to an independent group such as a group comprising similarly exercising or rehabilitating individuals. The user may variously access any one or more of exercise completion, degree of compliance to or conformation to a target or model reference data, change in compliance to a target or model, and self-reported pain level at any one or more of a set of pain reporting checkpoints.

Physical rehabilitation is in some cases difficult for a user to comply with because progression is not readily apparent. Patients may lose incentive to comply to a rehabilitation regimen because of a lack of awareness of or sense of accomplishment or failure to feel as if progress is occurring, or because of a feeling that progress has either plateaued or completed. By providing compliance data, particularly in the form of changes in degree of compliance to or conformation to a target or model reference data set, change in compliance to a target or model, and self-reported pain level at any one or more of a set of pain reporting checkpoints, a user may be able to visualize progression through a rehabilitation regimen so as to be incentivized to continue along the regimen even if the user does not feel or is not otherwise aware of progress or improvement.

That is, systems and methods herein enable a user to concurrently document movement improvement, such as movement improvement related to a orthobiologic insert or other medical composition, and pain management demand improvement. Such documentation may be provided to a user so as to incentivize compliance, for example by demonstrating progress or points of improvement to the user. Such documentation may be provided to a medical professional so as to allow assessment of a rehabilitation regimen, for example pursuant to modification of such a regimen or pursuant to assessment of the need for or efficacy of follow-on intervention. Such documentation may be provided to an orthobiologic manufacturer or provider, so as to assess efficacy of the orthobiologic, such documentation may be provided to a medical professional or an insurer, so as to assess user compliance or user need for additional surgery or pharmaceutical pain intervention.

Accordingly, a cohort of individuals using systems and methods herein in some cases exhibit a level of compliance to or rate of completion of a rehabilitation regimen that is at least 1%, 2%, 5%, 10%, 20%, 50% or greater than 50% more than the level of compliance to or rate of completion of a cohort of individuals not using the systems and methods herein, such as by performing rehabilitation on site in the presence of a medical practitioner.

Surgery independent movement and pain assessment. As mentioned above, systems and methods herein facilitate not only recovery from surgery, such as surgery relating to introduction of an orthobiologic or other medical composition or device, but also surgery-independent movement, alone or integrated with movement related pain assessment and alleviation, in some cases in a system to facilitate reporting to a medical provider such as a remote medical provider or other movement assessor, or to document movement for ongoing assessment.

That is, the systems and methods herein are in some cases used to assess movement or movement and self-reported pain in the contest of surgical assessment. A user may, for example, have suffered an acute event such as a fall, car accident or other skeletal or neurological trauma, or example, such that movement is impaired. Alternately, a user may suffer from a chronic or ongoing issue such as a herniated disk, spina bifida, multiple sclerosis or other disorder impacting movement.

Systems and methods herein facilitate movement assessment, alone or in combination with pain assessment, so as to facilitate degree of movement impact, either at a particular point or over time. Assessment may be done so as to compare a movement data set for an individual to either a healthy movement dataset, for example so as to detect deviation from a healthy movement dataset consistent with a particular disorder, such as loss of mobility at a particular disk or vertebra. Alternately or in combination, one may compare to one or more datasets consistent with particular disorders, such that alignment with a dataset indicates presence of the disorder associated with the dataset. Concurrently, self-reported pain assessment may indicate severity of impact on user quality of life, and efficacy of non-medicinal pain management to reduce self-reported pain levels may indicate the viability or likelihood of completion of a rehabilitation regimen.

Such an assessment may comprise or facilitate one or more of the following: rehabilitation selection, injury identification, orthobiologic or other medical composition identification, or surgery selection. Such assessment may comprise an individual assessment data set collection, or may comprise a temporal series of data collection events, so as to assess user progression over time. In some such cases, trends in performance over time such as one or more of decreasing deviation from healthy dataset positioning, increasing deviation from injury or disorder dataset positioning, improvement relative to original movement dataset, decrease in self-reported pain level, increase in responsiveness to alternative pain mediation may be assessed to provide patient condition or select a future course of action. Assessment is in some cases made by a medical practitioner to whom the dataset or datasets are reported, for example through a remote terminal functionality provided pursuant to a system herein. Alternately or in combination, assessment is made through artificial intelligence or other automated or computer or cloud-based data assessment.

Movement assessment may be performed in combination with or independent of injury or surgical intervention. For example, an athletic performance dataset comprising one or a temporal series of movement performances may be collected. This data may be compared to a dataset or datasets associated with model or correct movement, or with incorrect movement, or both correct and incorrect movement, so as to assess user technique or form in performing a motion, risk of injury in performing the motion, or change in the motion in response to coaching or practice. Exemplary motions in these embodiments include baseball pitching, such as pitching that may imperil elbow health or performed subsequent to elbow surgery, baseball batting, basketball free-throw or other shot taking, other throwing, golf ball or tennis ball striking or other activity comprising or involving elbow or spinal movement, balance exercises such as gymnastic performance, running such as sprinting or distance running, other track and field event performance, skiing, swimming or diving, or other sport or physical activity, dancing, acting or other activity performed by a professional or amateur.

Assessment of sport or other physical performance in some cases further comprises a self-reported pain assessment or in some cases a self-reported fatigue assessment, alone or made before and after a post-performance treatment such as icing, massage, or nonmedicinal pain management.

Data collected from such an assessment may be presented to a user, and or may be provided to a medical practitioner or athletic specialist such as a coach or trainer who may be remote to the user.

Data collection. A number of data collection approaches are consistent with the disclosure herein. Data are in some cases collected through a single data collection system such as a video recorder as is found in a handheld device. Alternate data collection systems further comprise markers such as joint markers to identify particular joints or portions of a user body or indicative of position or motion of the user.

Substantial data processing may be performed so as to identify particular joints or movements pursuant to user performance of an activity. Processing may comprise identifying joint position and joint movement pursuant to performance of a movement or series of movements. Identification may be aided by joint markers, or may be performed solely on a video or image of user movement. Some data collection approaches comprise one or more of detection of wearable motion sensors or position markers that are identified in images of user exercise performance by an image capture device. Alternatively, user body positions are identified without the aid of position markers or wearable sensors. The images are subjected to analysis to generate 2D joint estimation datasets, such as via a convolutional neural network or other modeling approach. Through the 2D joint estimation images, joint position probability is mapper and depth triangulation is performed so as to assign position to the joint positions.

In an exemplary CNN approach, 3D pose estimation and comparison to a reference is effected through one or more of the following steps. An individual wearing motion sensors performs a pose exercise regimen under the view of a video capture device such as a phone. The video images are transmitted to a cloud computing locale where they are subjected to analysis by a convolutional neural network, comprising 2D joint estimation based upon wearable motion sensor data alone, user image data alone, or a combination of the two. Joint probability is then mapped based upon the 2D estimation. The mapped joint probability data is subjected to depth triangulation or other analysis to generate three dimensional joint probability data that may be used as a three dimensional depiction or representation of a user pose which can readily be compared to a reference joint dataset, such that difference from or similarity to a reference can be calculated.

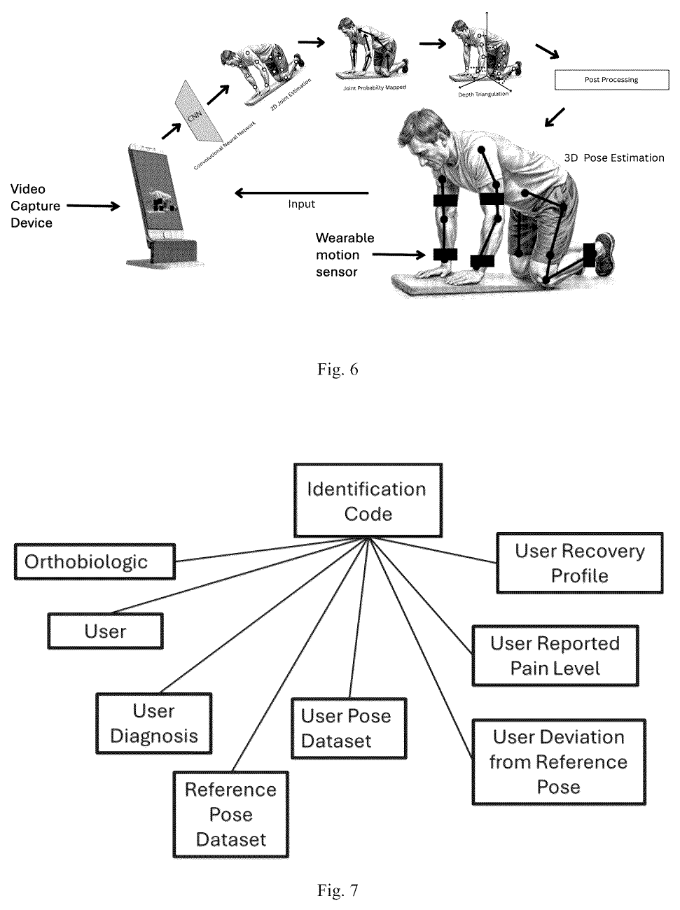

This dataset or a previous dataset in the processing workflow is subjected to post-processing, such as to compare to a target reference joint position dataset or a data set of pre-intervention or early post-intervention joint position, as shown in FIG. 6.

Data presentation. Data may be presented through any one of a spectrum of approaches.

A most direct data presentation approach comprises delivering positive or negative outcomes, such as ‘check,’ ‘plus’ or ‘+’ or ‘x’ for performance within a threshold deviation from a target, at a level of improvement, or outside of a threshold for a negative movement model. Alternately, percentage correlation or deviation from a reference dataset such as a target reference joint position dataset or a data set of pre-intervention or early post-intervention joint position. Such a system facilitates visualization of multiple measurement sessions or instances over time, such that a user or medical professional may observe successful compliance over time or changes in compliance or performance over time through multiple steps of a treatment regimen. The output may additionally be modified or multiply depicted to indicate pain level at one or more pain level assessment points. That is, a check for successful performance may be colored or bolded, for example to indicate self-reported pain level prior to, immediately after or subsequent to nonmedicinal pain intervention following performance of an instance of movement monitoring, or change in pain level following nonmedicinal pain management. Output may be additionally modified so as to incentivize compliance or continued performance, for example by indicating a duration of consecutive performance (a ‘streak’), or a duration at a particular pain level or attainment of a new pain level or progression through a gating to a new exercise in an exercise or therapy regimen.

More data rich presentation methods may also be used, particularly in presentation of movement data and pain level data to a medical practitioner. For example, data may depict a value representative of deviation from a reference, such as a healthy movement pattern, an injured or affected movement pattern, or a previous movement pattern data set of the user.

The data may further indicate which joints or which components of the movement pattern contribute to deviation, if any, from a model or reference data set. Some datasets may further depict or allow a user or a medical practitioner to select or observe a stored user movement video corresponding to a dataset or dataset component. In yet further embodiments, depictions corresponding to user joint or other body positions are highlighted or otherwise marked to indicate where in the completion of a movement or exercise the joint or body part deviates from a reference, such as a healthy movement pattern, an injured or affected movement pattern, or a previous movement pattern data set of the user.

The data may further indicate self-reported pain levels prior to or upon completion of a pose or movement pattern, as well as responsiveness of pain levels to a pain alleviation exercise. Accordingly, data may facilitate pain alleviation task selection or alteration so as to inform medical practitioner selection of a pain remediation or alleviation strategy.

### Pain Medication Reduction

Nonmedicinal pain reduction. Some systems and methods herein relate to replacement of medicinal pain treatment by using nonmedical pain amelioration or reduction approaches. Some systems comprise providing mental stimulation or mental distraction as an approach to pain reduction. Such mental stimulation may be effected through generation de novo or customization and provision of third-party generative art creation software or art or other puzzle activities, for example so as to engage a user mentally or otherwise distract a user from pain such as post movement pain.

Examples include drawing or coloring exercises, art-related puzzles, memorization exercises, mathematical puzzles, presenting optical illusion visual puzzles, music, text such as poetry, chanting recitations, philosophical or historically significant text, or riddles, or other distracting or engaging subject matter.

Nonmedical pain reduction is provided in response to movement or regimen portion completion followed by self-reported pain, in some cases above a threshold or alternately whenever such pain is present. In some cases, the complexity, elaborateness, volume, duration or other parameter of the nonmedical pain reduction is tailored to the magnitude of the self-reported pain. Alternately, pain reduction is provided in response to self-reported pain independent of the level reported.

Nonmedical pain reduction is provided alone or in combination with medicinal pain reduction in various embodiments. In some cases, efficacy of nonmedical pain reduction indicates a reduced need for medical pain reduction, such that medical pain reduction may be reduced or prescribed at a lower level or effected using an less potent component in response to observation of efficacy of nonmedical pain reduction, as measured for example by reduction in self-reported pain following nonmedical pain reduction therapy.

Various embodiments of art therapies or other alternate pain reduction therapies exhibit a pain reduction of at least, about, or no more than 5%, 10%, 15%, 20%, 25%, 30%, 35%, 40%, 45%, 50%, 55%, 6-%, 65%, 705, 75% 80%, 85%, 90%, 90%, or up to 100%, as measured by self-reported pain levels before and after an individual pain reduction therapy instance.

Similarly, various embodiments of art therapies or other alternate pain reduction therapies result in a reduction of use in medicinal pain reduction of at least, about, or no more than 5%, 10%, 15%, 20%, 25%, 30%, 35%, 40%, 45%, 50%, 55%, 6-%, 65%, 705, 75% 80%, 85%, 90%, 90%, or up to 100%, relative to a control individual or set of individuals undergoing a comparable regimen not using a method or system herein.

The accessibility of alternate pain reduction immediately after motion or exercise completion or cessation may reduce the time during which a user may experience post-exercise pain. Accordingly, practice of alternate pain reduction herein may reduce the overall pain associated with movement or exercise, so as to increase the chance of user compliance with a therapy regimen by, for example, 10%, 20%, 30%, 40%, 50%, 60%, 70%, 80%, 90%, or 100%. Practice of alternate pain reduction herein may increase the chance of user completion of a therapy regimen by, for example, 10%, 20%, 30%, 40%, 50%, 60%, 70%, 80%, 90%, or 100%. Practice of alternate pain reduction herein may increase user progress through a regimen at a set time period (at least, at most about or for example, 1, 2, 3, 4, 5, 6, 7, 8 weeks, 3, 4, 5, 6, months or more) after initiating a therapy regimen by, for example, 10%, 20%, 30%, 40%, 50%, 60%, 70%, 80%, 90%, or 100% or more. Practice of alternate pain reduction herein may increase user recovery at a set time period (at least, at most about or for example, 1, 2, 3, 4, 5, 6, 7, 8 weeks, 3, 4, 5, 6, months or more) after initiating a therapy regimen by, for example, 10%, 20%, 30%, 40%, 50%, 60%, 70%, 80%, 90%, or 100% or more. All measurements are variously relative to a control individual or set of individuals undergoing a comparable regimen not using an alternate pain reduction herein.

Like other methods herein, pain medication reduction is in some cases effected through use of the kit components described elsewhere herein, such that disclosure in those sections is informative of some embodiments of methods of performing these tasks. That is, pain self reporting or pain responsiveness to pain alleviation task completion may inform pain medication selection, such that pain medication use is reduced in light of lower pain reporting scores or greater responsiveness of pain to pain alleviation task completion, or increased in light of higher pain reporting scores or lower responsiveness of pain to pain alleviation task completion.

Informing gated regimen progression. Upon completion of a nonmedical pain reduction exercise, user pain level may be again self-reported so as to again measure pain levels. Levels or changes in self-reported pain levels may inform the level of stress the movement exercise placed on the user, and may inform progression through a gated therapeutic movement regimen, or selection of which from a plurality of options for progression. Progression decisions are in some cases made through artificial intelligence or other automated analysis through systems herein or based on algorithms set based in score number, percent increase or other direct measurement. Alternately, decisions are made by a medical professional to whom results are reported, such as remotely, alone or informed by recommendations provided through artificial intelligence or other automated analysis. In some cases decisions are made by a user, such as a user informed by data collected through use of the methods and kits herein, and as may be presented to the user so as to facilitate assessment.

Progression decisions variously comprise gating advancement through a linear course, selecting among a first and a second option for progression through a bifurcated course, decisions to remain at a current stage of a regimen because of pain levels independent of user movement data positive or negative correlation to a reference model, increasing or decreasing rest intervals between movement exercises, or other moderation or modulation of regimen progression.

Like other methods herein, gated progression is in some cases effected through use of the kit components described elsewhere herein, such that disclosure in those sections is informative of some embodiments of methods of performing these tasks.

Monitoring of self-reporting. Through systems and methods herein, user pain self-reporting is monitored, such as prior to movement recordation, subsequent to movement recordation, and subsequent to undergoing nonmedical pain reduction. Pain levels may be analyzed, such as through automated or AI driven analysis, and trends reported to the user or to a healthcare practitioner, such as a remote healthcare practitioner.

Data may be analyzed for trends between movement recording sessions or among reportings related to a single movement session or both between sessions and among reportings as part of a single session. Analysis outcomes variously comprise informing gating regimen progression, informing medicinal pain reduction selection or dosage, informing nonmedical pain reduction exercise selection or duration, or informing decisions related to surgical progression or orthobiologic selection of surgical progression is selected.

Action thresholds. In various embodiments, a pain gated action threshold is passed if a pre-exercise self-reported pain level remains at no more than 95%, 90%, 80%, 70%, 60%, 50%, or less, for example, of a prior self-reported pain level or of a maximum pain level for 1, 2, 3, 4, 5, 6, 7, 8, 9, 10 or more than 10 exercise or movement sessions. Similarly, in various embodiments, a pain gated action threshold is passed if a post-exercise self-reported pain level remains at no more than 95%, 90%, 80%, 70%, 60%, 50%, or less, for example, of prior self-reported pain level or of a maximum pain level for 1, 2, 3, 4, 5, 6, 7, 8, 9, 10 or more than 10 exercise or movement sessions, or at least or no more than 100%, 110%, 120%, 130%, 140%, 150% or greater than 150% of a pre-exercise self-reported pain level for a given session. Similarly, in various embodiments, a pain gated action threshold is passed if a post-nonmedical pain reduction exercise self-reported pain level remains at no more than 95%, 90%, 80%, 70%, 60%, 50%, or less, for example, or prior self-reported pain level or of a maximum pain level for 1, 2, 3, 4, 5, 6, 7, 8, 9, 10 or more than 10 exercise or movement sessions, or remains at no more than 95%, 90%, 80%, 70%, 60%, 50%, or less, for example, of a pre-exercise or post-exercise self-reported pain level for a given exercise or movement instance. Action thresholds may inform gating regimen progression, informing medicinal pain reduction selection or dosage, informing nonmedical pain reduction exercise selection or duration, or informing decisions related to surgical progression or orthobiologic selection of surgical progression is selected.

### Synergy of Pose Estimation and Pain Management or Other Art Therapy

Some methods herein comprise a component of synergistic intertwining of data collected and processed from the Pose-Estimation and Pain Management such as Art Therapy components into a composite assessment, which may be used to extrapolate insights that consider both physical therapy adherence and Art Therapy adherence for individual pain improvement.

The composite assessment enables the software application to recommend alternative Art Therapy or other pain management pathways in the case that physical therapy adherence has been confirmed by the software or by a medical professional without an improvement in the pain score over a set period, such as over a number of sessions or a period of time, for example, 1, 2, 3, 4 5, 6, or 7, or more days, or 1, 2, 3, 4 or more than 4 weeks, 1, 2, or more than 2 months, for example over two week intervals.

Similarly, the composite assessment enables the software application to recommend alternative Physical Therapy regimen session pathways in the case that pain therapy adherence or progress has been confirmed by the software or by a medical professional without an improvement in the physical therapy session performance over a set period, such as over a number of sessions or a period of time, for example, 1, 2, 3, 4 5, 6, or 7 or more days, or 1, 2, 3, 4 or more than 4 weeks, 1, 2, or more than 2 months, for example over two week intervals.

Similarly, the composite assessment enables the software application or medical practitioner to recommend concurrent alternative Physical Therapy regimen session pathways and alternative Pain remediation pathways in the case of system confirmed or system identified and medical professional confirmed lack of improvement in either physical therapy performance or pain remediation or both.

Patients may also be provided with a comparative score of their Physical Therapy adherence in relation to their Art Therapy adherence.

Like other methods herein, synergy of pose performance and pain alleviation is in some cases effected through use of the kit components described elsewhere herein, such that disclosure in those sections is informative of some embodiments of methods of performing these tasks.

### User and Medical Practitioner Reporting

Systems and methods herein have a broad range of functionalities. In addition to measuring and documenting user movement and evaluating movement relative to a reference, and documenting, ameliorating and analyzing self-reported pain levels, and in some cases analyzing user data to make recommendations, some systems and methods may present, analyze and facilitate analysis of movement or exercise data by a user or medical practitioner, such as a remote medical practitioner, such that system analyses and decisions or recommendations may be independently assessed. Similarly, some systems comprise a functionality such as an application to allow medical practitioner to remotely access user data.

Consistent with these functionalities, some systems comprise an interface or interfaces, for example for a user, a medical practitioner, or both a user and a medical practitioner may access system or method data. Such interface or interfaces are in some cases remote monitors or application or computer functionalities to facilitate data access, such as real time access or temporally remote access to user data.

Thus, systems and methods herein facilitate remote assessment by a medical practitioner, such that the medical practitioner does not need to be physically present at user movement performance or exercise completion, or the user does not need to be present at a medical practitioner office during at user movement performance or exercise completion. Images or videos are nonetheless captured and available for the medical practitioner, and accompanying data analysis and movement evaluation can be accessed, without a medical practitioner being present during user movement performance or exercise completion.

Furthermore, some systems and methods propose courses of action, such as user progression through to a next stage of a movement regimen, selection among at least one of a plurality of second stages of a movement regimen, such as in response to one or more of user movement performance relative to a reference, self-reported pain evaluation, or self-reported pain response to nonmedical or medical treatment, or other inputs, or selection of pain alleviation approaches. These courses of action are in some cases proposed independently of a medical practitioner, or serve as a gating of progression through a computationally composed or medical practitioner developed movement or movement and pain modulation regimen, or are proposed for approval or assessment by a medical practitioner who makes a final assessment as to gating or progression decisions. Often, in combination, systems and methods provide a medical practitioner with user data, such as movement reference matching or divergence data, information as to which joints or other body parts or movements result in divergence from a reference movement data set, relation of patient movement to orthobiologic insert or other medical composition position and movement predictions, user movement movies, user self-reported pain assessment prior to or subsequent to movement performance, or prior to and subsequent to pain intervention such as medicinal or nonmedicinal pain intervention. This information facilitates medical practitioner assessment of user performance and of system automated recommendations as to movement regimen progression.

Consequently, regimen progression recommendations are in some cases made rapidly, such as immediately after user movement completion of pain alleviation completion, or more rapidly or more accurately by a medical practitioner than in the absence of such systems and methods.

Thus, a user may practice or execute a movement performance or exercise without needing to schedule or be in the presence of a medical practitioner, while nonetheless not losing the benefit of the information being available to the medical practitioner, and while also gaining the benefit of automated or Al analysis of movement performance or exercise completion, sometimes in real time, and rapid assessment and regimen progression recommendations.

This rapid, convenient integration of exercise information and recommendations has a huge effect on user compliance with a rehabilitation or other physical therapy regimen. A user may perform movement or exercise tasks at her or his convenience rather than scheduling and in some cases traveling to a remote site. Results are immediately accessible to the user and in a format that is easily assessable or digestible by the user, while at the same time being available to a medical practitioner. Nonmedicinal pain reduction is available immediately after movement or exercise completion, without prescription or delay, as is analysis of pain status or efficacy of both the exercise regimen and the nonmedicinal pain reduction approach. At the same time, information and gated progression recommendations are available to a medical practitioner, such as a remote medical practitioner, to facilitate rapid analysis of user performance and approval of user progression through a rehabilitation or other exercise regimen, including both direct progression and user performance specific modification of either movement or exercise progression, pain treatment approach, or both movement and pain approaches.

This ease of access and performance of movement exercises interacts synergistically with the ease of presentation of user progress, both in movement and in pain reduction, in providing pain amelioration, and in the recordation of ‘streaks’ and other progress benchmarks, so as to incentivize user compliance and ultimately user recovery.

Accordingly, practice of a method or use of a system herein may increase the chance of user compliance with a therapy regimen by, for example, at least 10%, 20%, 30%, 40%, 50%, 60%, 70%, 80%, 90%, or 100%. Practice of a method or use of a system herein may increase the chance of user completion of a therapy regimen by, for example, at least 10%, 20%, 30%, 40%, 50%, 60%, 70%, 80%, 90%, or 100%. Practice of a method or use of a system herein may increase user progress through a regimen at a set time period (for example, 1, 2, 3, 4, 5, 6, 7, 8 weeks, 3, 4, 5, 6, months or more) after initiating a therapy regimen by, for example, at least 10%, 20%, 30%, 40%, 50%, 60%, 70%, 80%, 90%, or 100% or more. Practice of a method or use of a system herein may increase user recovery at a set time period (for example, 1, 2, 3, 4, 5, 6, 7, 8 weeks, 3, 4, 5, 6, months or more) after initiating a therapy regimen by, for example, at least 10%, 20%, 30%, 40%, 50%, 60%, 70%, 80%, 90%, or 100% or more. All measurements are variously relative to a control, such as a control individual or set of individuals undergoing or having undergone a comparable regimen not using a method or system herein.

### Identification Code-Based Data Aggregation

Consistent with the kits, compositions and methods herein, disclosed herein are methods related to coordination of data related to rehabilitation pursuant to surgical or other intervention comprising use of an orthobiologic, other biologic, or other medical device or composition by assigning or associating the data with an identification code assigned to the orthobiologic, other biologic, or other medical device or composition. In some cases the identification code is initially assigned to the orthobiologic, other biologic, or other medical device or composition by being affixed as a barcode or other information source to packaging such as packaging that maintains sterility of the packaged contents. Alternately, some identification codes may be encoded within the identified object, such as through a chip, or otherwise associated with the object. In many cases, a barcode on the object is scanned pursuant to a surgical intervention, and scanning is in some cases performed concurrent with release of the object from packaging pursuant to a surgical intervention, though earlier and later identification code acquisition are also consistent with the disclosure herein.

Subsequent to selection of the object for use in a medical intervention on a user or recipient of the object and association of the identification code with the object and in some cases with biochemical characteristics of the object, the identification code is associated with user information, such as one or more of user identity, user condition or diagnosis, user medical intervention to be performed, timing of intervention, user pre-intervention reference movement data, user pre-intervention pain level data, or other information available prior to or during surgery.

Pursuant to rehabilitation, the identification is further associated with data generated thereby. For example, user exercise or pose performance or performance related positional data, analysis thereof that may lead to joint position data, reference data, analysis results arising from pose data such as user divergence or difference from pose reference data such as reference pose data or user prior pose data, user pose performed and timing, user rehabilitation pose progression status, self-reported pain data, pain alleviation exercises performed, efficacy of pain alleviation exercise as measured by pain self-reporting levels after performance of pain alleviation, for example, pain or other medication administered or taken by the user and its impact on pain levels and pose performance, among other data.

Accordingly, identification codes herein facilitate user data consolidation by anchoring or associating object data and user data to an object identification code that is available to a surgeon or other medical practitioner at the time of application of the object to the user. As user data is further generated, it is readily associated with the identification code so as to facilitate consolidated data assembly and streamlined data analysis, so as in some cases to facilitate real-time progression recommendations subsequent to pose performance or pain alleviation task completion, and ease of data presentation to a medical practitioner such as a medical practitioner remote from the user who may access the data associated with the identification code through a computer implemented physician portal, such as one governed by a physician portal software package as may be available in a kit to which the object to be administered to the user is bundled, or other kit as disclosed elsewhere herein.

As used herein, the term “about” in the context of a number refers to a range spanning from 10% less than to 10% greater than that number, while in the context of a range refers to a larger range spanning from 10% below the lower listed limit to 10% greater than the upper listed limit of the range.

Turning to the Figures, one sees the following.

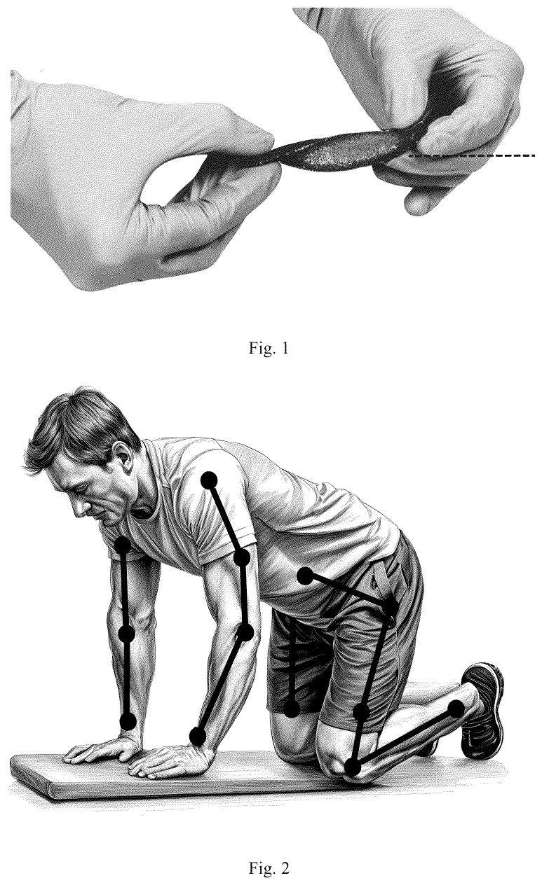

At FIG. 1, one sees a drawing of an orthobiologic material consistent with the disclosure herein. The orthobiologic material is being twisted to demonstrate its strength and flexibility. The material is wetted with bone marrow aspirate. The combination of resilience and cell absorbency is attributable to the composition of embedded granules, bioactivated silica and the collagen matrix in which they reside. The orthobiologic is assigned an identifying or unique mobile activation code that enrolls the patient or user in a convolutional neural network-powered physical therapy program after surgery, allowing for a link from surgery to post-operative recovery and art-centered pain management.

At FIG. 2, one sees a user performing a prescribed physical therapy exercise as prompted by the mobile application. The convolutional neural network underpinning the mobile application is extrapolating the user's joint positions, indicated by the darkened circles and lines, so as to assess user performance of the exercise in real time, and to record completion of an exercise. The mobile application then is able in some cases to recommend a subsequent course of progression through a multistep exercise regimen.

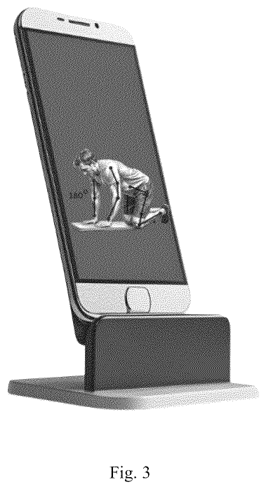

At FIG. 3, one sees a mobile device consistent with the disclosure herein. The device in some cases captures images or a movie of the user performing the exercise regimen, and assesses performance. Physical therapy adherence and progress is visible on the user's mobile device. The user is able to see her or his own performance, and divergence or discrepancies between user performance and model performance are highlighted in real time. This allows the user to adjust performance during an exercise session rather than upon receiving feedback subsequent to performance. The performance of each individual exercise repetition, as well as the number of exercises performed, and the degree of divergence from a model are recorded and the data made available to the user and to a medical practitioner for evaluation. Upon completion of an exercise, recommendations as to progression through an exercise regimen are made to the user or for approval by a medical practitioner having access to the data.

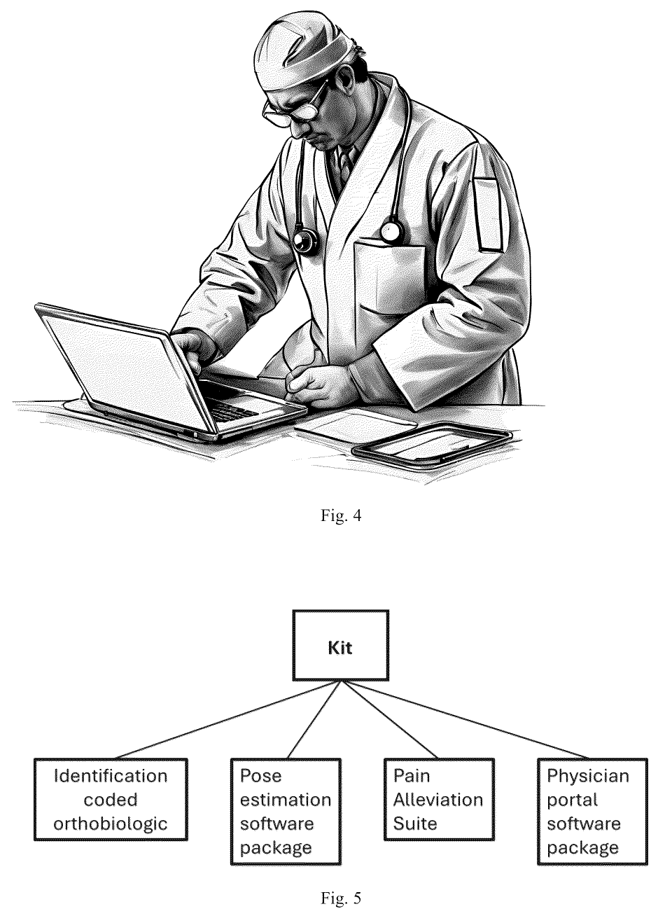

At FIG. 4, one sees that a medical practitioner can access the user's physical therapy and art therapy adherence information, both for a particular exercise and for a course of exercises over time. The data is accessed using a secure portal that includes data such as graphs of progress, a therapy regimen multi-exercise course based upon the orthobiologic lot number, with proposed next step exercises. The secure portal allows the medical practitioner to access user performance data in real time or subsequent to user performance, remote from the site of user exercise performance, such that the user does not need to schedule a medical practitioner or travel to a medical practitioner site to perform an exercise. Similarly, the medical practitioner does not need to physically observe the user in order to assess user performance or deviation from a healthy or successful performance of an exercise. Rather, the medical practitioner may rely upon joint-mapped data collected and annotated by the system and presented at the portal, system assessments of user performance, or actual images of the user. Secure portals are in some cases hardware, or may be an application or applications that a medical practitioner may access on a computer or other computational device.

At FIG. 5, one sees a schematic of a kit consistent with the disclosure herein. The kit in the figure comprises an identification coded implantable orthobiologic, a pose estimation software package, a pain alleviation suite and a physician portal software package as described elsewhere herein bundled into a single kit. Various kits consistent with the disclosure herein comprise some or all of the elements depicted herein and may further comprise additional components.

At FIG. 6, one sees detection of a set of wearable motion sensors by a video capture device such as that of FIG. 3, and analysis of the input via onboard computational capacity or communication of this data to a computer cloud for cloud-based assessment.

As shown therein, a user wearing a motion sensor set or positional marker set is performing a rehabilitation pose. The motion sensors, depicted as sold black boxes, are attached to the user's wrists, upper arms, and lower calves in this depiction, though a number of attachment points are consistent with the disclosure herein. Only the wrist motion sensor is labeled. The sensors provide motion sensing data that is conveyed as input to a video capture device such as that of FIG. 3. The video capture device in some cases comprises a computational capacity to execute pose estimation on the input, while in other cases transmits the input to a remote processor, such as a cloud based processor, having computational capacity to execute pose estimation on the input. The processor analyzes the input, such as using a convolutional neural network or other computational approach, to correlate wearable motion sensor and in some cases image input data to generate a joint estimation dataset, which may be a 2D or 3D joint estimation dataset. This joint estimation is them used to map joint probability, to which depth triangulation is performed to generate a 3D point position matrix or dataset, which may be compared to a reference to assess user exercise performance.

At FIG. 7 one sees a schematic of an identification code consistent with the kits and methods herein. The code, which may be a barcode or other code attached to an orthobiologic or other medical composition or device as disclosed herein or which may be indicative of information other than an orthobiologic, as in cases where methods involving nonsurgical pose evaluation are used. The code is in many cases attached to a sealed orthobiologic package, and may be scanned pursuant to opening the package during surgery or preparation for surgery. The identification code is in various embodiments associated with one or more of the orthobiologic insert or other medical composition or device, the user or recipient, a diagnosis or condition associated with the user, and a reference pose dataset, such as a model pose dataset or user pre-intervention dataset, or other information available at the time of a surgical intervention. Pursuant to use, the identification code may be associated with or have assigned to it data related to a user rehabilitation pose dataset, data related to user deviation from a reference pose dataset, user reported pain level prior to user rehabilitation pose performance, subsequent to pose performance, prior to pain alleviation task performance, subsequent to pain alleviation task performance, a user recovery profile, medical practitioner recommendations or other information relevant to user recovery or use of kits herein.

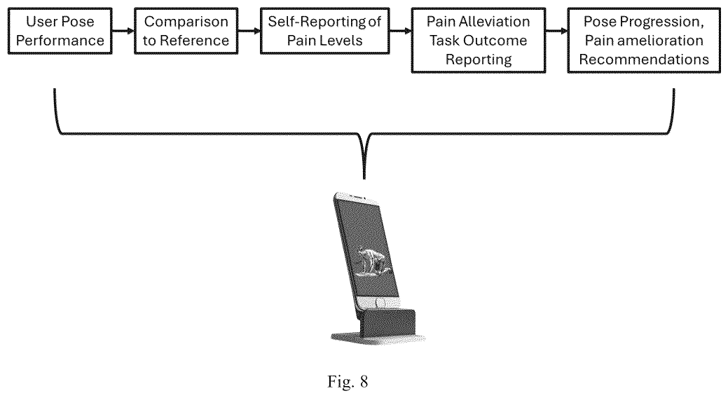

At FIG. 8, one sees a pose progression workflow consistent with the kits, systems and methods herein. A user performs a pose or other exercise, either pursuant to rehabilitation following s surgical intervention such as one that introduces an orthobiologic into the user, or pursuant to nonsurgical intervention or nom-medical pose evaluation such as athletic or artistic performance training. One or more of the following are then used to assess user progression. User pose or exercise data are captured and subjected to analysis such as comparison to a reference, for example a target dataset or a baseline dataset, and deviation for the reference is calculated. User self-reported pain levels, or self-reported fatigue levels, are reported.

An assessment is then generated as to user pose progression. The assessment variously comprises consideration of one or more of the factors mentioned immediately above or elsewhere throughout the present disclosure. The assessment comprises a recommendation as to whether the user should continue with the present pose exercise, progress to a more advanced pose, progress to a localized exercise joint or body-part performance defect-specific pose exercise, return to a previous pose exercise, progress to one among a plurality of pose progression alternatives, increase repetitions or intensity for a current pose exercise, or otherwise advise as to exercise or rehabilitation course. Alternatively or in combination, the assessment comprises a recommendation or a selection as to user pain amelioration strategy. That is, a recommendation in some cases comprises a recommendation or selection as to whether the user should continue with the same pain amelioration exercise or type of exercise, increase level of difficulty or complexity, increase duration of the pain amelioration, or change the type of pain amelioration mental exercise.

In some cases, a pain assessment comprises recommendation or selection of a pharmaceutical treatment or a change in pharmaceutical treatment to accompany a rehabilitation regimen. That is, in some cases an assessment comprises a recommendation as to adding, ceasing, or increasing or decreasing dosage of a pharmaceutical pain management composition, such as an over the counter or prescription pain medicine. The recommendation is presented to a medical practitioner or, as appropriate, provided directly to the user.

Similarly, an assessment may select or recommend a pose or exercise that directs motion away from or deemphasizes, or alternatively increases motion or stress at a localized pain point, such as a joint or orthobiologic insertion site, so as to manage pain or challenge the user in a rehabilitation course.

The assessment is performed on a computer, such as a cloud computing platform, or medical practitioner or user computer or handheld computing device such as a phone, as depicted in the figure. In some cases the assessment is preformed substantially immediately after receipt of data, or without substantial delay, such that a user may access the assessment conclusions upon completion of the pose or pain assessment or pain alleviation exercise or post pain alleviation exercise pain assessment. That is, the user does not need to wait for a medical practitioner appointment to access results. Similarly, a medical practitioner, such as one accessing the results from a remote portal, may similarly receive recommendations or assessment promptly upon accessing the user provided results.

### Numbered Embodiments

The technology is further understood in light of the following partial list of numbered embodiments. 1. A kit comprising an implantable orthobiologic, a pose estimation software package, a pain alleviation suite, and a physician portal software package. 2. The kit of any numbered embodiment, such as number 1, wherein the pose estimation package comprises an interface for a mobile image capture device. 3. The kit of any numbered embodiment, such as number 1, wherein the pose estimation package comprises an interface for detection of positional markers on a user. 4. The kit of any numbered embodiment, such as number 3, wherein the user is a surgical recipient of the implantable orthobiologic. 5. The kit of any numbered embodiment, such as number 1, wherein the pose estimation package comprises at least one user positional marker. 6. The kit of any numbered embodiment, such as number 1, wherein the pose estimation package comprises an interface for detection of a wearable motion sensor. 7. The kit of any numbered embodiment, such as number 6, wherein the wearable motion sensor is a third party manufactured wearable motion sensor. 8. The kit of any numbered embodiment, such as number 1, wherein the pose estimation package comprises a wearable motion sensor. 9. The kit of any numbered embodiment, such as number 1, wherein the pose estimation package comprises a kinetic model for at least one rehabilitation exercise. 10. The kit of any numbered embodiment, such as number 1, wherein the pose estimation package identifies at least one anatomical landmark in a user motion dataset. 11. The kit of any numbered embodiment, such as number 10, wherein the user motion dataset comprises at least one video frame. 12. The kit of any numbered embodiment, such as number 11, wherein the at least one video frame is captured by a third party video capture device. 13. The kit of any numbered embodiment, such as number 10, wherein the user motion dataset comprises at least one position monitor data array. 14. The kit of any numbered embodiment, such as number 10, wherein the pose estimation package identifies the at least one anatomical landmark using a deep learning model. 15. The kit of any numbered embodiment, such as number 14, wherein the deep learning model comprises a machine learning library. 16. The kit of any numbered embodiment, such as number 15, wherein the machine learning library comprises a Pytorch learning library. 17. The kit of any numbered embodiment, such as number 14, wherein the deep learning model is validated against at least one annotated reference dataset. 18. The kit of any numbered embodiment, such as number 17, wherein the pose estimation software package comprises at least one annotated reference dataset, and wherein the pose estimation software package generates an evaluation of positional information deviation from the at least one annotated reference dataset. 19. The kit of any numbered embodiment, such as number 18, wherein the at least one annotated reference dataset represents a healthy individual pose. 20. The kit of any numbered embodiment, such as number 18, wherein the at least one annotated reference dataset represents a pre-intervention individual pose. 21. The kit of any numbered embodiment, such as number 18, wherein the at least one annotated reference dataset represents a user pre-intervention pose. 22. The kit of any numbered embodiment, such as number 18, wherein the at least one annotated reference dataset corresponds to a data set involving the implantable orthobiologic. 23. The kit of any numbered embodiment, such as number 18, wherein the pose estimation package records completion of an exercise when the deviation is below a threshold. 24. The kit of any numbered embodiment, such as number 23, wherein the pose estimation package displays completion of the exercise. 25. The kit of any numbered embodiment, such as number 18, wherein the kit coveys the evaluation to a medical practitioner using the physician portal software package. 26. The kit of any numbered embodiment, such as number 25, wherein the medical practitioner is remote from the user. 27. The kit of any numbered embodiment, such as number 10, wherein the pose estimation package displays an image comprising the at least one anatomical landmark. 28. The kit of any numbered embodiment, such as number 27, wherein the anatomical landmark corresponds to a site of implantation of the implantable orthobiologic. 29. The kit of any numbered embodiment, such as number 27, wherein the anatomical landmark does not correspond to a site of implantation of the implantable orthobiologic. 30. The kit of any numbered embodiment, such as number 1, wherein the pose estimation package interfaces with a computer cloud based assessment capacity. 31. A kit comprising an orthobiologic, a computer implemented pose estimation software package, a computer implemented pain alleviation suite, and a computer implemented physician portal software package. 32. The kit of any numbered embodiment, such as number 31, wherein the orthobiologic is an implantable orthobiologic. 33. The kit of any numbered embodiment, such as number 32, wherein the implantable orthobiologic comprises a tag. 34. The kit of any numbered embodiment, such as number 32, wherein the implantable orthobiologic comprises bioactive glass. 35. The kit of any numbered embodiment, such as number 32, wherein the implantable orthobiologic comprises beta-tricalcium phosphate. 36. The kit of any numbered embodiment, such as number 32, wherein the implantable orthobiologic comprises hydroxyapatite. 37. The kit of any numbered embodiment, such as number 32, wherein the implantable orthobiologic comprises collagen. 38. The kit of any numbered embodiment, such as number 37, wherein the collagen is bovine collagen. 39. The kit of any numbered embodiment, such as number 32, wherein the implantable orthobiologic mimics kit recipient bone. 40. The kit of any numbered embodiment, such as number 32, wherein the implantable orthobiologic is consistent with healing achieved through use of a patient autologous bone graft. 41. The kit of any numbered embodiment, such as number 33, comprising at least one sticker corresponding to the tag. 42. The kit of any numbered embodiment, such as number 33, comprising a hermetically sealed packaging having an identification code corresponding to the tag. 43. The kit of any numbered embodiment, such as number 33, wherein the tag is scannable. 44. The kit of any numbered embodiment, such as number 31, wherein the computer implemented pose estimation package interfaces with a mobile image capture device. 45. The kit of any numbered embodiment, such as number 31, wherein the computer implemented pose estimation package interfaces with positional markers on a user. 46. The kit of any numbered embodiment, such as number 31, wherein the computer implemented pose estimation package interfaces with a wearable motion sensor. 47. The kit of any numbered embodiment, such as number 31, wherein the computer implemented pose estimation package comprises a wearable motion sensor. 48. The kit of any numbered embodiment, such as number 45, wherein the user is a surgical recipient of the implantable orthobiologic. 49. The kit of any numbered embodiment, such as number 31, wherein the computer implemented pose estimation package comprises a kinetic model for at least one rehabilitation exercise. 50. The kit of any numbered embodiment, such as number 31, wherein the computer implemented pose estimation package identifies at least one anatomical landmark in a user motion dataset. 51. The kit of any numbered embodiment, such as number 50, wherein the user motion dataset comprises at least one video frame. 52. The kit of any numbered embodiment, such as number 50, wherein the user motion dataset comprises at least one position monitor data array. 53. The kit of any numbered embodiment, such as number 50, wherein the computer implemented pose estimation package identifies the at least one anatomical landmark using a deep learning model. 54. The kit of any numbered embodiment, such as number 53, wherein the deep learning model comprises a machine learning library. 55. The kit of any numbered embodiment, such as number 54, wherein the machine learning library comprises a Pytorch learning library. 56. The kit of any numbered embodiment, such as number 53, wherein the deep learning model is validated against at least one annotated reference dataset. 57. The kit of any numbered embodiment, such as number 56, wherein the computer implemented pose estimation package evaluates positional information deviation from at least one annotated reference dataset. 58. The kit of any numbered embodiment, such as number 50, wherein the computer implemented pose estimation package displays an image comprising the at least one anatomical landmark. 59. The kit of any numbered embodiment, such as number 50, wherein the computer implemented pose estimation package records completion of an exercise. 60. The kit of any numbered embodiment, such as number 59, wherein the computer implemented pose estimation package displays completion of the exercise. 61. The kit of any numbered embodiment, such as number 31, wherein the computer implemented pain alleviation suite receives a first user pain rating. 62. The kit of any numbered embodiment, such as number 61, wherein the first user pain rating is user self-reported. 63. The kit of any numbered embodiment, such as number 62, wherein the first user pain rating is provided prior to the pain alleviation suite generating an output. 64. The kit of any numbered embodiment, such as number 61, wherein the user pain rating is informed at least in part by an assessment of deviation of user positional information deviation from at least one annotated reference dataset. 65. The kit of any numbered embodiment, such as number 61, wherein the computer implemented pain alleviation suite recommends a pain treatment. 66. The kit of any numbered embodiment, such as number 65, wherein the pain treatment comprises computer implemented generative art creation software. 67. The kit of any numbered embodiment, such as number 66, wherein the computer implemented generative art creation software provides a colorable art template. 68. The kit of any numbered embodiment, such as number 66, wherein the computer implemented generative art creation software provides a visual puzzle. 69. The kit of any numbered embodiment, such as number 65, wherein the pain treatment comprises a pain medication. 70. The kit of any numbered embodiment, such as number 69, wherein the pain treatment is proposed to an observing medical professional. 71. The kit of any numbered embodiment, such as number 66, wherein the computer implemented pain alleviation suite receives a second user pain rating subsequent to the pain treatment. 72. The kit of any numbered embodiment, such as number 71, wherein the computer implemented pain alleviation suite assesses effectiveness of the pain treatment. 73. The kit of any numbered embodiment, such as number 31, wherein the computer implemented physician portal software package monitors a user's pain score over time. 74. The kit of any numbered embodiment, such as number 31, wherein the computer implemented physician portal software package monitors a user's change in positional information deviation from at least one annotated reference dataset over time. 75. The kit of any numbered embodiment, such as number 31, wherein the computer implemented physician portal software package monitors a user's physical therapy schedule adherence over time. 76. The kit of any numbered embodiment, such as number 31, wherein the computer implemented physician portal software package displays a user pain treatment recommendation. 77. The kit of any numbered embodiment, such as number 31, wherein the computer implemented physician portal software package is accessible through the internet. 78. The kit of any numbered embodiment, such as number 31, wherein the computer implemented physician portal software package is accessible on a mobile internet device. 79. The kit of any numbered embodiment, such as number 31, wherein the computer implemented physician portal software package allows physician user communication. 80. The kit of any numbered embodiment, such as number 31, wherein a computer implemented community interface package. 81. The kit of any numbered embodiment, such as number 80, wherein the computer implemented community interface package facilitates performance data exchange between a first user and a second user. 82. A method of encouraging compliance to a recovery regimen, the method comprising digitally monitoring a user first physical therapy performance, digitally assessing divergence in user first physical therapy performance from a first physical therapy performance model performance, receiving a user first pain assessment, providing a user art based pain management exercise, and receiving a user second pain assessment. 83. The method of any numbered embodiment, such as number 82, wherein the recovery regimen is an orthobiologic intervention. 84. The method of any numbered embodiment, such as number 82, wherein the recovery regimen is a surgical intervention. 85. The method of any numbered embodiment, such as number 82, wherein the recovery regimen is a physical therapy intervention. 86. The method of any numbered embodiment, such as number 82, wherein digitally monitoring comprises video recording. 87. The method of any numbered embodiment, such as number 82, wherein digitally monitoring comprises recording user position monitoring marker location. 88. The method of any numbered embodiment, such as number 82, wherein digitally assessing comprises applying a deep learning model. 89. The method of any numbered embodiment, such as number 88, wherein the applying a deep learning model comprises using a machine learning library. 90. The method of any numbered embodiment, such as number 89, wherein the machine learning library corresponds to a position monitoring output associated with an implantable orthobiologic delivered to the user. 91. The method of any numbered embodiment, such as number 82, wherein the art based pain management comprises drawing. 92. The method of any numbered embodiment, such as number 82, wherein the art based pain management comprises puzzle solving. 93. The method of any numbered embodiment, such as number 82, comprising comparing the divergence in user first physical therapy performance to the user first pain assessment. 94. The method of any numbered embodiment, such as number 82, comprising comparing the divergence in user first physical therapy performance to the user second pain assessment. 95. The method of any numbered embodiment, such as number 82, comprising reporting at least one of the divergence in user first physical therapy performance, first user pain assessment and second user pain assessment to the user. 96. The method of any numbered embodiment, such as number 95, wherein the reporting comprises indicating a value for at least one previous physical performance of at least one of the divergence in user previous physical therapy performance, previous first user pain assessment and previous second user pain assessment to the user. 97. The method of any numbered embodiment, such as number 95, wherein the reporting comprises indicating a value for at least five previous physical performances of at least one of the divergence in user previous physical therapy performance, previous first user pain assessment and previous second user pain assessment to the user. 98. The method of any numbered embodiment, such as number 97, wherein the report comprises improvement for at least one previous physical performance of at least one of the divergence in user previous physical therapy performance, previous first user pain assessment and previous second user pain assessment to the user. 99. The method of any numbered embodiment, such as number 82, comprising reporting at least one of the divergence in user first physical therapy performance, first user pain assessment and second user pain assessment to a medical practitioner. 100. The method of any numbered embodiment, such as number 99, wherein the medical practitioner is remote to the user. 101. The method of any numbered embodiment, such as number 82, comprising reporting at least one of the divergence in user first physical therapy performance, first user pain assessment and second user pain assessment to a user community. 102. A method comprising providing a movement exercise to a user, generating a record of the user performing the movement exercise, and assessing divergence between the recording and a reference performance of the movement exercise. 103. The method of any numbered embodiment, such as number 102, comprising reporting divergence between the recording and a reference performance of the movement exercise to the user. 104. The method of any numbered embodiment, such as number 102, comprising reporting divergence between the recording and a reference performance of the movement exercise to a medical practitioner. 105. The method of any numbered embodiment, such as number 104, wherein the medical practitioner is remote to the user. 106. The method of any numbered embodiment, such as number 102, wherein the movement exercise comprises a rehabilitation exercise. 107. The method of any numbered embodiment, such as number 106, wherein the rehabilitation exercise is targeted to prevent surgical intervention. 108. The method of any numbered embodiment, such as number 106, wherein the rehabilitation exercise is targeted to facilitate recovery from surgical intervention. 109. The method of any numbered embodiment, such as number 108, wherein the surgical intervention comprises introduction of an orthobiologic to the user. 110. The method of any numbered embodiment, such as number 106, wherein the rehabilitation exercise is targeted to prevention of a harm to the user. 111. The method of any numbered embodiment, such as number 102, wherein the movement exercise comprises an athletic training exercise. 112. The method of any numbered embodiment, such as number 102, wherein assessing divergence comprises performing a computational analysis of the user performing the movement exercise. 113. The method of any numbered embodiment, such as number 112, wherein the computational analysis comprises artificial intelligence analysis of the user performing the movement exercise. 114. The method of any numbered embodiment, such as number 102, wherein assessing divergence comprises identifying user body parts responsible for the divergence. 115. The method of any numbered embodiment, such as number 102, wherein assessing divergence comprises identifying user joints responsible for the divergence. 116. The method of any numbered embodiment, such as number 102, wherein assessing divergence comprises identifying user movement subcomponents responsible for the divergence. 117. The method of any numbered embodiment, such as number 102, wherein the reference is a healthy performance of the movement exercise. 118. The method of any numbered embodiment, such as number 102, wherein the reference is a prior user performance of the movement exercise. 119. The method of any numbered embodiment, such as number 102, comprising receiving a self reported pain level from the user prior to performing the movement exercise. 120. The method of any numbered embodiment, such as number 102, comprising receiving a self reported pain level from the user subsequent to performing the movement exercise. 121. The method of any numbered embodiment, such as number 102, comprising providing to the user a nonmedicinal pain management exercise subsequent to the user performing the movement exercise. 122. The method of any numbered embodiment, such as number 121, wherein the providing is immediately subsequent to the user performing the movement exercise. 123. The method of any numbered embodiment, such as number 122, comprising receiving a self reported pain level from the user subsequent to performing the nonmedicinal pain management exercise. 124. The method of any numbered embodiment, such as number 102, comprising receiving a self reported pain level from the user prior to performing the movement exercise, receiving a self reported pain level from the user subsequent to performing the movement exercise, providing to the user a nonmedicinal pain management exercise subsequent to the user performing the movement exercise, and comprising receiving a self reported pain level from the user subsequent to performing the nonmedicinal pain management exercise. 125. The method of any numbered embodiment, such as number 102, comprising generating a report of the assessing. 126. The method of any numbered embodiment, such as number 102, comprising generating a report of the assessing and of the patient self reported pain levels from at least one of prior to performing the movement exercise, subsequent to performing the movement exercise and subsequent to performing the nonmedicinal pain management exercise. 127. The method of any numbered embodiment, such as number 126, wherein the report comprises an assessment of whether the user performance constitutes a substantial match to the reference. 128. The method of any numbered embodiment, such as number 127, wherein the substantial match comprises at least 80% match to the reference performing. 129. The method of any numbered embodiment, such as number 125, wherein the report comprises degree of divergence from the reference. 130. The method of any numbered embodiment, such as number 125, wherein the report indicates extent of compliance to a temporal regimen of movement performance exercises. 131. The method of any numbered embodiment, such as number 125, wherein the report is provided to the user. 132. The method of any numbered embodiment, such as number 125, wherein the report is provided to a medical practitioner. 133. The method of any numbered embodiment, such as number 132, wherein the medical practitioner is remote to the user. 134. The method of any numbered embodiment, such as number 126, comprising assessing contents of the report to select a progression course through a movement exercise regimen. 135. The method of any numbered embodiment, such as number 134, wherein assessing contents of the report to select a progression course through a movement exercise regimen comprises selecting a next movement exercise when user performance constitutes a substantial match to the reference. 136. The method of any numbered embodiment, such as number 135, wherein the next movement exercise is selected from a linear exercise regimen. 137. The method of any numbered embodiment, such as number 136, wherein the linear exercise regimen is provided by a medical practitioner. 138. The method of any numbered embodiment, such as number 134, wherein assessing contents of the report to select a progression course through a movement exercise regimen comprises selecting a next movement exercise when user performance constitutes a substantial match to the reference. 139. The method of any numbered embodiment, such as number 134, wherein assessing contents of the report to select a progression course through a movement exercise regimen comprises selecting a next movement exercise to target a user joint when user performance differs from the reference related to movement at the user joint. 140. The method of any numbered embodiment, such as number 135, wherein assessing contents of the report to select a progression course through a movement exercise regimen comprises selecting a next movement exercise when at least one user self reported pain level is below a threshold. 141. The method of any numbered embodiment, such as number 134, comprising reporting results of the assessing to a medical practitioner. 142. The method of any numbered embodiment, such as number 141, wherein the medical practitioner is remote to the user. 143. A method of encouraging compliance to a movement regimen, comprising providing a nonmedicinal pain management exercise to a user immediately subsequent to user performance of a movement exercise of the exercise regimen, providing a performance assessment to the user immediately subsequent to user performance of a movement exercise of the exercise regimen, and providing the performance assessment to a medical practitioner. 144. The method of any numbered embodiment, such as number 143, wherein the medical practitioner is remote to the user. 145. The method of any numbered embodiment, such as number 143, wherein the user exhibits compliance corresponding to adherence to the exercise regimen better than at least 50% of users provided the exercise regimen without being provided the nonmedicinal pain management exercise immediately subsequent to performance. 146. The method of any numbered embodiment, such as number 143, wherein the user exhibits compliance corresponding to adherence to the exercise regimen better than at least 50% of users provided the exercise regimen without being provided the performance assessment immediately subsequent to performance. 147. The method of any numbered embodiment, such as number 143, wherein the user exhibits compliance corresponding to adherence to the exercise regimen better than at least 50% of users provided the exercise regimen without providing the performance assessment to a medical practitioner. 148. A method of selecting a physical mobility intervention course, the method comprising digitally monitoring a user first physical performance, digitally assessing divergence in user first physical performance from a first physical performance model performance, receiving a user first pain assessment, and selecting an implantable orthobiologic to introduce into the first user. 149. The method of any numbered embodiment, such as number 148, wherein digitally monitoring comprises video recording. 150. The method of any numbered embodiment, such as number 148, wherein digitally monitoring comprises recording user position monitoring marker location. 151. The method of any numbered embodiment, such as number 148, wherein digitally assessing comprises applying a deep learning model. 152. The method of any numbered embodiment, such as number 151, wherein the applying a deep learning model comprises using a machine learning library. 153. The method of any numbered embodiment, such as number 152, wherein the machine learning library corresponds to at least one position monitoring output associated with a defect addressable using an implantable orthobiologic delivered to the user. 154. The method of any numbered embodiment, such as number 148, wherein selecting an implantable orthobiologic comprises weighing user first pain assessment and divergence in user first physical performance from a first physical performance model performance. 155. The method of any numbered embodiment, such as number 153, wherein selecting an implantable orthobiologic comprises identifying an implantable orthobiologic to address the defect. 156. The method of any numbered embodiment, such as number 155, wherein selecting an implantable orthobiologic comprises assessing user first pain assessment. 157. A kit comprising a pose estimation software package, a pain alleviation suite, and a physician portal software package. 158. The kit of any numbered embodiment, such as number 157, comprising a wearable sensor. 159. The kit of any numbered embodiment, such as number 157, comprising an implantable orthobiologic. 160. An implantable orthobiologic, comprising a porous biocompatible matrix, and a population of composite granules. 161. The orthobiologic of any numbered embodiment, such as number 160, wherein the matrix comprises collagen. 162. The orthobiologic of any numbered embodiment, such as number 160, wherein the matrix comprises dispersed bioactive glass particles. 163. The orthobiologic of any numbered embodiment, such as number 160, wherein the matrix exhibits a porosity interconnectivity of at least 70%. 164. The orthobiologic of any numbered embodiment, such as number 160, wherein the matrix exhibits a porosity interconnectivity of at least 80%. 165. The orthobiologic of any numbered embodiment, such as number 160, wherein the matrix exhibits a porosity interconnectivity of 80%-90%. 166. The orthobiologic of any numbered embodiment, such as number 160, wherein the matrix comprises pores of at least 350um diameter. 167. The orthobiologic of any numbered embodiment, such as number 160, wherein the matrix comprises pores of a diameter ranging from 500 um to 600 um. 168. The orthobiologic of any numbered embodiment, such as number 160, wherein the matrix comprises pores of a diameter ranging from 530 um to 570 um. 169. The orthobiologic of any numbered embodiment, such as number 160, wherein the granules are embedded in the matrix. 170. The orthobiologic of any numbered embodiment, such as number 160, wherein the granules exhibit a higher density than the matrix. 171. The orthobiologic of any numbered embodiment, such as number 160, wherein the granules comprise a resorption component. 172. The orthobiologic of any numbered embodiment, such as number 171, wherein the resorption component comprises at least 50% of the granule for each granule. 173. The orthobiologic of any numbered embodiment, such as number 171, wherein the resorption component comprises at least 90% of the granule for each granule. 174. The orthobiologic of any numbered embodiment, such as number 171, wherein the resorption component comprises about 95% of the granule for each granule. 175. The orthobiologic of any numbered embodiment, such as number 171, wherein the resorption component comprises beta-tricalcium phosphate. 176. The orthobiologic of any numbered embodiment, such as number 160, wherein the granules comprise a structural component. 177. The orthobiologic of any numbered embodiment, such as number 176, wherein the structural component comprises no more than 50% of the granule for each granule. 178. The orthobiologic of any numbered embodiment, such as number 176, wherein the structural component comprises no more than 20% of the granule for each granule. 179. The orthobiologic of any numbered embodiment, such as number 176, wherein the structural component comprises about 5% of the granule for each granule. 180. The orthobiologic of any numbered embodiment, such as number 176, wherein the structural component comprises hydroxyapatite. 181. The orthobiologic of any numbered embodiment, such as number 160, wherein the orthobiologic comprises an imbibed cell population. 182. The orthobiologic of any numbered embodiment, such as number 181, wherein the imbibed cell population comprises mesenchymal cells. 183. The orthobiologic of any numbered embodiment, such as number 181, wherein the imbibed cell population comprises cells from a bone marrow aspirate.

### EXAMPLES

Example 1. Orthobiologic. An orthobiologic insert is synthesized for insert into a user. The orthobiologic is synthesized to have a trabecular structure similar to cancellous bone. The orthobiologic exhibits an 85% interconnected porosity among pores having a median diameter of 550 um. The orthobiologic is synthesized from a composition comprising a collagen framework and a plurality of particles. One subset of the particles comprises composite granules of 95% beta-tricalcium phosphate and 5% hydroxyapatite, a molar ratio of 61:1. Another subset of the particle comprises bioactivated glass.

The orthobiologic is delivered in hermetically-sealed packaging with a distinguishing identification code, which is printed on the package and on removable stickers. The code is used to identify the orthobiologic in a motion analysis and pain amelioration system that is provided with the orthobiologic, such that orthobiologic or the target region to which it is directed can be used to select an intervention-appropriate physical therapy regimen that is provided as a proposal to a medical practitioner.

Example 2. Orthobiologic replacement by user bone or collagen tissue. The orthobiologic of Example 1 is prepared for delivery into a user. The delivery process comprises harvesting user osteoblasts derived from a user mesenchymal stem cell population derived from bone marrow aspirate obtained concurrent with surgery, and imbibing them into the orthobiologic. As the osteoblasts are on average 20-30 um in diameter, the orthobiologic porosity allows them to both spread through and attach to the orthobiologic scaffold.

The orthobiologic is delivered to the user insertion site subsequent to imbibing, such that incorporation of user bone forming cells into orthobiologic is effected prior to delivery.

This process continues subsequent to insertion, facilitated by the orthobiologic porosity and by the granule composition. The beta-tricalcium phosphate is gradually resorbed by user tissue to be replaced by user bone or collagen formation, while the hydroxyapatite and the bioactivated glass is persistent and serves to maintain orthobiologic structure.

Example 3. Surgical intervention kit. A hospital obtains a surgical intervention kit pursuant to surgery. The kit comprises an orthobiologic insert and access to support technology. The support technology includes a rehabilitation regimen comprising multiple exercises suitable to rehabilitation subsequent to surgical insertion of the orthobiologic. The support technology also includes motion capture functionalities such that a user performing a rehabilitation regimen may be recorded and her or his performance of one or more rehabilitation routines may be captured and assessed as to its compliance to or deviation from a reference dataset. Assessment variously comprises identification of user positions such as joint positions, limb positions or posture.

Dataset references are provided by the kit, such as previously generated healthy or incrementally improved reference datasets. Alternately or in combination, references are generated through kit technology from prior user rehabilitation capture datasets.

The kit further comprises a reporting functionality, such that the user may receive a real time assessment of rehabilitation regimen performance relative to the reference, and such that a medical practitioner may also receive the assessment.

The kit further comprises a recommendation functionality, such that the kit provides a recommendation as to whether the user should continue with a current rehabilitation exercise or to progress to one or more alternate rehabilitation exercises informed by deviation from or compliance with a reference. The recommendation is submitted to the medical practitioner for independent assessment.

The kit further comprises a pain management functionality by which the user is provided with a pain management activity subsequent to performance of a rehabilitation exercise. The pain management activity may be an art project, a puzzle, or other mentally engaging activity that may redirect the user's attention from pain, such as pain associated with the rehabilitation exercise, with the surgical intervention, or with the issue necessitating the surgical intervention.

The kit comprises an interface so that it may receive a user first pain self assessment prior to performance of the pain management activity and a second pain self assessment subsequent to performance of the pain management activity. In some cases the user first pain assessment, second pain assessment or both first and second pain assessment are used by the kit to inform the recommendation functionality.

The reporting functionality is also used to provide the user first pain assessment and second pain assessment to the medical practitioner, who may use it to inform rehabilitation regimen progression decisions.

The orthobiologic is introduced to the user via a surgical intervention, and is labeled so that the kit may identify the user and correlate the user to a rehabilitation regimen tailored to the orthobiologic, the issue necessitating the intervention and to the user.

The technological components of the kit are conveyed to the user via electronic communication, such that the user may communicate with the technologies of the kit, such as by sending rehabilitation exercise data to the kit and receiving rehabilitation exercise evaluation and pain management recommendations from the kit.

A communication interface is further communicated to a medical practitioner, such that the medical practitioner may receive exercise performance information, pain management information and regimen progression recommendations, and may approve or propose regimen next steps.

Example 4. A user receives access to a kit of Example 3 pursuant to a surgery introducing the orthobiologic of the kit. Access is emailed to the user, and an image capture functionality is added to the user's personal image capture device, such as a phone.

The user receives an exercise regimen proposal, and performs the exercise under the watchful eye of his phone camera. The data are transferred to the kit where user joints and body postures are identified and assessed relative to a reference.

The user is found to deviate from the reference, and the user reports a high pain level. The kit provides a pain management activity comprising a picture to be colored in. The user reports a reduced pain level subsequent to performance of the activity without pharmaceutical intervention, thus reducing the risk of pain medication addiction associated with recovery.

The kit proposes a second exercise to address defects identified in the user's performance of the first exercise and to address the user's high reported post-exercise pain level. The second exercise is communicated to a remote medical practitioner, who approves the second exercise.

The user performs the second exercise and observes an increase in performance data compliance to the healthy reference, and decreased reported pain subsequent to performance.

The kit proposes that the user return to the first exercise, which is approved by a remote medical practitioner.

The user returns to the first exercise and performs the second exercise with a lower deviation from a reference dataset and lower reported pain, which is managed by a post-exercise pain management activity.

The user them progresses through to a third exercise toward complete rehabilitation from the surgical intervention.

Example 5. Data association with an orthobiologic identification code. A user is identified as having an issue to be addressed using an orthobiologic kit as disclosed herein. The kit comprises an orthobiologic insert in a barcoded hermetically sealed package. During surgery, shortly before administration of the orthobiologic to the user, the barcode is scanned, the packaging opened and the orthobiologic prepared for administration to the user by cellular infusion, prior to administration to the user.

The orthobiologic, chemical characteristics of the infusion, the user, the user diagnosis, timing of the surgery, pre-surgery motion capture data of the user to serve as a baseline reference, and pre-surgery self-reported pain levels are associated with the information code.

Subsequent to surgery, the user embarks upon a rehabilitation course comprising a series of pose exercises. Upon performance of a pose, pose images are captured facilitated by position markers worn by the user. A pose model is created for the user, and compared to a reference pose model dataset to evaluate user progression through rehabilitation. Subsequent to pose completion, the user performs a pain alleviation suite exercise so as to non-pharmaceutically reduce pain levels.

Pose performance data, pose deviation from a reference, self-reported pain levels prior to and subsequent to pain alleviation performance, timing of completion of the pose and other relevant data are recorded and associated with the identification code, as are recommendations as to progression through the pose exercise regimen and as to pain alleviation.

A medical practitioner enters the identification code into a remote portal, and is able to access all of the data associated with the identification code, such as orthobiologic insert identity, diagnosis, time since surgery, previous and current pose performance relative to a reference, pain levels, and an automated assessment as to next steps in progression. The medical practitioner assesses the data associated with the identification code and responds to the automated assessment, entering the response so that it is associated with the identification code. The medical practitioner is able to respond quickly, with all available data and images, without being present at user performance of the pose, and the user is able to access the medical practitioner recommendations associated with the identification code, without having to travel or perform the pose exercise on site in the presence of the medical practitioner.

Example 6. Conventional rehabilitation. A user undergoes an insert surgery and is provided with a rehabilitation regimen. The regimen comprises poses to be performed in view of a medical practitioner, so as to evaluate progression.

The user schedules appointments with the medical practitioner and travels to the medical practitioner site to perform the pose exercises.

Example 7. Rehabilitation using the disclosure herein. A user undergoes an insert surgery and is provided with a rehabilitation regimen associated with an identification code as part of a kit comprising the orthobiologic, software for image capture, software for exercise performance assessment, pain alleviation mental exercises, software for progress assessment and software for a remote portal functionality. The regimen comprises poses to be performed in view of an image capture device, for remote analysis and transmission to a medical practitioner portal for evaluation.

The user performs pose exercises and pain alleviation remote from the medical practitioner, without scheduling appointments or traveling to a medical practitioner site. Image data is captured, pain assessment s saved, and progression recommendations are made and provided to the medical practitioner through a remote portal. The user is able to perform rehabilitation exercises at the user's own schedule without traveling to a medical practitioner suite, and subsequently performs rehabilitation exercises much more frequently, receiving automated assessments promptly thereafter.

The medical practitioner receives all current and prior user data, as well as automated assessments and recommendations, facilitating rapid assessment or approval of automated assessments.

The user consequently performs substantially more rehabilitation exercises in a given time, and progresses more rapidly though the regimen than under conventional conditions as in Example. 6.

1. A method of assessing performance of a recovery regimen, the method comprising digitally monitoring a user first physical therapy performance, digitally assessing divergence in user first physical therapy performance from a first physical therapy performance reference performance, receiving a user first pain assessment, providing a user mental pain management exercise, and receiving a user second pain assessment.

2. The method of claim 1, wherein the recovery regimen is an orthobiologic intervention recovery regimen.

3. The method of claim 1, wherein the recovery regimen is a surgical intervention recovery regimen.

4. The method of claim 1, wherein the recovery regimen is a physical therapy intervention.

5. The method of claim 1, wherein digitally monitoring comprises video recording.

6. The method of claim 1, wherein digitally monitoring comprises recording user position monitoring marker location.

7. The method of claim 1, wherein digitally assessing comprises applying a deep learning model.

8. The method of claim 7, wherein the applying a deep learning model comprises using a machine learning library.

9. The method of claim 8, wherein the machine learning library corresponds to a position monitoring output associated with an implantable orthobiologic delivered to the user.

10. The method of claim 1, wherein the mental pain management comprises drawing.

11. The method of claim 1, wherein the mental pain management comprises puzzle solving.

12. The method of claim 1, comprising comparing the divergence in user first physical therapy performance to the user first pain assessment.

13. The method of claim 1, comprising comparing the divergence in user first physical therapy performance to the user second pain assessment.

14. The method of claim 1, comprising reporting at least one of the divergence in user first physical therapy performance, first user pain assessment and second user pain assessment to the user.

15. The method of claim 14, wherein the reporting comprises indicating a value for at least one previous physical performance of at least one of the divergence in user previous physical therapy performance, previous first user pain assessment and previous second user pain assessment to the user.

16. The method of claim 14, wherein the reporting comprises indicating a value for at least five previous physical performances of at least one of the divergence in user previous physical therapy performance, previous first user pain assessment and previous second user pain assessment to the user.

17. The method of claim 16, wherein the report comprises improvement for at least one previous physical performance of at least one of the divergence in user previous physical therapy performance, previous first user pain assessment and previous second user pain assessment to the user.

18. The method of claim 1, comprising reporting at least one of the divergence in user first physical therapy performance, first user pain assessment and second user pain assessment to a medical practitioner remote to the user.

19. The method of claim 1, comprising reporting at least one of the divergence in user first physical therapy performance, first user pain assessment and second user pain assessment to a user community

20. The method of claim 1, wherein the recovery regimen is assigned an identification code, the identification code being associated with a diagnosis of the user, the first physical therapy performance reference performance, the user first physical therapy performance, the user mental pain management exercise, the user first pain assessment, and the user second pain assessment.
